(19) United States
(12) Patent Application Publication
(10) Pub. No.: US 2012/0197141 A1
(43) Pub. Date: Aug. 2, 2012

(54) IMPLANTABLE ECHO DOPPLER FLOW SENSOR FOR MONITORING OF HEMODYNAMICS

(75) Inventors: Guy Vanney, Blaine, MN (US); Thao Ngo, Shakopee, MN (US); Scott Sjoquist, Minnetonka, MN (US); Dorab N. Sethna, Los Angeles, CA (US); Annapurna Karicherla, Valencia, CA (US); George K. Lewis, Andover, MA (US); Dan E. Gutfinger, Agoura Hills, CA (US); Gene A. Bornzin, Simi Valley, CA (US)

(73) Assignee: PACESETTER, INC., Sylmar, CA (US)

(21) Appl. No.: 13/016,101

(22) Filed: Jan. 28, 2011

(51) Int. Cl. A61B 5/026

(52) U.S. Cl. 600/505

(57) ABSTRACT

Systems, devices and methods of monitoring blood flow velocity are disclosed herein. For example, one method of monitoring blood flow velocity includes: locating a blood flow velocity sensor near the ostium in the coronary sinus; and sensing towards a portion of the aorta. A second example method includes: locating a blood flow velocity sensor in a vein; and sensing towards an adjacent artery. A third example method includes: locating a blood flow velocity sensor near the tricuspid valve; and sensing towards a tricuspid valve annulus. A fourth example method includes: locating a blood flow velocity sensor right ventricular outflow tract; and sensing towards a portion of the aorta. A fifth example method includes: locating a blood flow velocity sensor in the great cardiac vein; and sensing towards a left anterior descending artery. A sixth example method includes: locating a blood flow velocity sensor in the right atrial appendage; and sensing towards a portion of the aorta.

### FIELD OF THE INVENTION

The present invention relates to medical devices and methods. More specifically, the present invention relates to devices and methods of monitoring hemodynamics.

### BACKGROUND OF THE INVENTION

Before a physician can provide appropriate therapy for a patient experiencing one or more hemodynamically compromising events, the physician will need to diagnose the event(s). For example, the physician may need to determine if the patient's event(s) is/are ischemia, atrial tachycardia (“AT”), atrial fibrillation (“AF”), ventricular tachycardia (“VT”), ventricular fibrillation (“VF”), inappropriate timing of atrial and ventricular pacing (“A-V timing”), and/or inappropriate timing of left ventricle and right ventricle pacing (“V-V timing”). The physician may also need to distinguish supra-ventricular tachycardia (“SVT”) from ventricular tachycardia (“VT”) and/or other hemodynamically compromising events.

There is a need in the art for systems, devices and methods that facilitate the diagnosis of hemodynamically compromising events.

### BRIEF SUMMARY OF THE INVENTION

Disclosed herein is a medical device. In one embodiment, the device includes a tubular body and a blood flow velocity sensor. The tubular body includes a distal end and a segment proximal to the distal end that is biased to assume a curved configuration. The sensor is supported on the segment. The tubular body is part of an implantable medical lead, a catheter or sheath.

A method of monitoring blood flow velocity is also disclosed herein. In one embodiment, the method includes: providing a medical device comprising: a tubular body including a distal end and a segment proximal to the distal end, the segment being capable of biasing into a curved configuration; and a blood flow velocity sensor supported on the segment, wherein the tubular body is part of an implantable medical lead, a catheter or sheath; delivering the tubular body into a patient such that the sensor is located in a volume of a first venous, arterial or cardiac structure; orienting the sensor to sense in the direction of a volume of a second venous, arterial or cardiac structure; and allowing the segment to bias into the curved configuration to at least temporarily secure the sensor orientation.

Disclosed herein is another medical device. In one embodiment, the device includes a tubular body, a fixation assembly, and a blood flow velocity sensor. The tubular body includes a distal end. The fixation assembly is near the distal end and includes a member deflectable away from the tubular body. The blood flow velocity sensor is near the fixation assembly. The tubular body is part of an implantable medical lead, a catheter or sheath.

Another method of monitoring blood flow velocity is disclosed herein. In one embodiment, the method includes: providing a medical device comprising: a tubular body including a distal end; a fixation assembly near the distal end, the fixation assembly including a member deflectable away from the tubular body; and a blood flow velocity sensor near the fixation assembly, wherein the tubular body is part of an implantable medical lead, a catheter or sheath; delivering the tubular body into a patient such that the sensor is located in a volume of a first venous, arterial or cardiac structure; orienting the sensor to sense in the direction of a volume of a second venous, arterial or cardiac structure; and causing the member to deflect away from the tubular body to at least temporarily secure the sensor orientation.

An implantable medical stent is also disclosed herein. In one embodiment, the stent includes an expandable body and a blood flow velocity sensor supported on the body.

Another method of monitoring blood flow velocity is disclosed herein. In one embodiment, the method includes: providing an implantable medical stent comprising: an expandable body; and a blood flow velocity sensor supported on the body; delivering the stent into a patient such that the sensor is located in a volume of a first venous, arterial or cardiac structure; orienting the sensor to sense in the direction of a volume of a second venous, arterial or cardiac structure; and causing the stent to expand to at least temporarily secure the sensor orientation.

Yet another medical device is disclosed herein. In one embodiment the medical device includes a tubular body, a deflection member and a blood flow velocity sensor. The tubular body includes a distal end. The deflection member extends longitudinally with the tubular body. Longitudinal displacement of the deflection member relative to the tubular body causes a segment of the tubular body to deflect into a curved configuration near the distal end. The blood flow velocity sensor is supported on the segment. The tubular body is part of an implantable medical lead, a catheter or sheath.

Yet another method of monitoring blood flow velocity is disclosed herein. In one embodiment, method includes: providing a medical device comprising: a tubular body including a distal end; a deflection member extending longitudinally with the tubular body, wherein longitudinal displacement of the deflection member relative to the tubular body causes a segment of the tubular body to deflect into a curved configuration near the distal end; and a blood flow velocity sensor supported on the segment, wherein the tubular body is part of an implantable medical lead, a catheter or sheath; delivering the tubular body into a patient such that the sensor is located in a volume of a first venous, arterial or cardiac structure; orienting the sensor to sense in the direction of a volume of a second venous, arterial or cardiac structure; and causing the tubular body to deflect into the curved configuration to at least temporarily secure the sensor orientation.

An implantable medical lead is disclosed herein. In one embodiment, the lead includes a tubular body, a suture sleeve and a blood flow velocity sensor. The tubular body includes a distal end. The suture sleeve is on the tubular body. The blood flow velocity sensor is near the suture sleeve.

Another method of monitoring blood flow velocity is disclosed herein. In one embodiment, the method includes providing a medical device comprising: a tubular body including a distal end; a suture sleeve on the tubular body; and a blood flow velocity sensor near the suture sleeve; delivering the tubular body into a patient such that the sensor is located in a volume of a first venous or arterial structure; orienting the sensor to sense in the direction of a volume of a second venous or arterial structure; and securing the suture sleeve to the patient to secure the sensor orientation.

Yet additional methods of monitoring blood flow velocity are disclosed herein. A first example method includes: locating a blood flow velocity sensor near the ostium in the coronary sinus; and sensing towards a portion of the aorta. A second example method includes: locating a blood flow velocity sensor in a vein; and sensing towards an adjacent artery. A third example method includes: locating a blood flow velocity sensor near the tricuspid valve; and sensing through the tricuspid valve annulus. A fourth example method includes: locating a blood flow velocity sensor in or near the right ventricular outflow tract; and sensing towards a portion of the aorta. A fifth example method includes: locating a blood flow velocity sensor in the great cardiac vein; and sensing towards the left anterior descending artery. A sixth example method includes: locating a blood flow velocity sensor in the right atrial appendage; and sensing towards a portion of the aorta.

Yet another medical device is disclosed herein. In one embodiment, the device includes a tubular body, and an echo Doppler sensor. The tubular body includes a distal end. The echo doppler sensor is supported on the tubular body and includes: an acoustic transmission side; another side opposite the acoustic transmission side; a piezoelectric sensor; a titanium housing forming a side wall about the piezoelectric sensor and located between the acoustic transmission side and the another side; and a low acoustic impedance material on an opposite side of the piezoelectric sensor from the acoustic transmission side. The tubular body is part of an implantable medical lead, a catheter or sheath.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an anterior view of the patient's upper chest region with the heart shown in partial cross section and illustrating the vasculature, a flow sensor being positioned in the subclavian vein peripheral to the subclavian artery and sensing towards the subclavian artery.

FIG. 2 is a graphical representation of electrocardiogram (“ECG”), echo doppler sensor (“Echo Sensor”) and aortic pressure (“Aortic Press.”) data taken when the patient's heart is functioning normally and the pressure sensor is positioned as discussed with respect to FIG. 1.

FIG. 3 is a graphical representation of electrocardiogram (“ECG”), echo doppler sensor (“Echo Sensor”) and aortic pressure (“Aortic Press.”) data taken when the patient's heart is undergoing ventricular fibrillation (“VF”) and recovery therefrom, the pressure sensor being positioned as discussed with respect to FIG. 1.

FIG. 4 is an anterior view of the heart shown in partial cross section, a flow sensor being positioned in the right atrium near the coronary sinus ostium and sensing towards the aortic root.

FIG. 5 is a graphical representation of surface electrocardiogram (“Surf ECG”), echo doppler (“Echo”), left ventricular pressure (“LVP”), aortic pressure (“AorticP”), right ventricular pressure (“RVP”), and Left Ventricular Volume (“Vtot”) versus Aortic Flow and Mitral Flow when the patient's heart is functioning normally and a flow sensor is positioned at the location discussed with respect to FIG. 4.

FIG. 6 is an anterior view of the heart shown in partial cross section, a flow sensor being positioned in the tricuspid annulus and sensing across the tricuspid annulus.

FIG. 7 is a graphical representation of surface electrocardiogram (“Surf ECG”), echo doppler (“Echo”), left ventricular pressure (“LVP”), aortic pressure (“AorticP”), right ventricular outflow tract pressure (“RVOT”), and Left Ventricular Volume (“Vtot”) versus Distinct E and A waves and RVOT Flow when the patient's heart is functioning normally and a flow sensor is positioned at the location discussed with respect to FIG. 6.

FIG. 8 is an anterior view of the heart shown in partial cross section, a flow sensor being positioned in the coronary sinus ostium and sensing towards the mitral annulus.

FIG. 9 is a graphical representation for intrinsic rhythm of surface electrocardiogram (“Surf ECG”), echo doppler (“Echo”), left ventricular pressure (“LVP”), aortic pressure (“AorticP”), right ventricular pressure (“RVP”), and Left Ventricular Volume (“Vtot”) versus Aortic Flow and Mitral Flow when the patient's heart is functioning normally and a flow sensor is positioned at the location discussed with respect to FIG. 8.

FIG. 10 is an anterior view of the heart, a flow sensor being positioned in the great cardiac vein and sensing towards the left anterior descending artery.

FIG. 11 is a graphical representation of surface electrocardiogram (“Surf ECG”), echo doppler (“Echo”), left ventricular pressure (“LVP”), aortic pressure (“AorticP”), right atrial pressure (“RA P”), and Left Ventricular Volume (“Vtot”) when the patient's heart is functioning normally and a flow sensor is positioned at the location discussed with respect to FIG. 10.

FIG. 12 is an anterior view of the heart, a flow sensor being positioned in the RVOT and sensing towards the aorta.

FIG. 13 is a graphical representation for intrinsic rhythm of electrocardiogram (“ECG”), echo Doppler (“Sensor”), left ventricular pressure (“LV P”), aortic pressure (“Aortic P”), and Left Ventricular Volume (“LV VOL”) when the patient's heart is functioning normally and a flow sensor is positioned at the location discussed with respect to FIG. 12.

FIG. 14 is an anterior view of the heart, a flow sensor being positioned in the RA appendage and sensing towards the aorta.

FIG. 15 is a graphical representation for intrinsic rhythm of electrocardiogram (“ECG”), echo doppler (“Sensor”), left ventricular pressure (“LV P”), aortic pressure (“Aortic P”), Left Ventricular Volume (“LV VOL”), and Echo Sound Recording (“EchoSnd”) when the patient's heart is functioning normally and a flow sensor is positioned at the location discussed with respect to FIG. 14.

FIG. 16 is an anterior view of the heart, a flow sensor being positioned in the superior vena cava and sensing towards the ascending aorta.

FIG. 17 is a graphical representation for intrinsic rhythm of electrocardiogram (“ECG”), echo Doppler (“Sensor”), left ventricular pressure (“LV P”), aortic pressure (“Aortic P”), and Left Ventricular Volume (“LV VOL”) when the patient's heart is functioning normally and a flow sensor is positioned at the location discussed with respect to FIG. 16.

FIG. 18 is a graphical representation for VT to VF transition of electrocardiogram (“ECG”), echo doppler (“Sensor”), left ventricular pressure (“LV P”), aortic pressure (“Aortic P”), Left Ventricular Volume (“LV VOL”), and Echo Sound Recording (“EchoSnd”) when the patient's heart is functioning normally and a flow sensor is positioned at the location discussed with respect to FIG. 16.

FIG. 19 is an isometric view of a lead distal end having a passive fixation configuration and having an echo Doppler sensor supported on the lead body in a manner that positions the sensor as discussed with respect to FIG. 4.

FIG. 20 is an isometric view of a lead distal end having an active fixation configuration and having an echo Doppler sensor supported on the lead body in a manner that positions the sensor as discussed with respect to FIG. 4.

FIG. 21 is a side view of an example of a lead based platform on which an echo Doppler sensor may be supported to position the sensor as discussed with respect to FIG. 6.

FIG. 22 is an isometric view of an example of a lead based platform on which an echo Doppler sensor may be supported to position the sensor as discussed with respect to FIG. 16.

FIG. 23 is a side view of an example of a lead based platform on which an echo Doppler sensor may be supported to position the sensor as discussed with respect to FIG. 8 or 10.

FIG. 24 is a side view of an example of a lead based platform on which an echo Doppler sensor may be supported to position the sensor as discussed with respect to FIG. 6 or 16.

FIG. 25 is a side view of an example of a lead based platform on which an echo Doppler sensor may be supported to position the sensor as discussed above with respect to FIG. 1.

FIG. 26 is a side view of an example of a lead or catheter based platform on which an echo Doppler sensor may be supported to position the sensor as discussed with respect to FIG. 4, 8, 10, 12, 14 or 16.

FIG. 27 is a side view of an example of a lead or catheter based platform on which an echo Doppler sensor may be supported to position the sensor as discussed with respect to FIG. 4, 8, 10, 12, 14 or 16.

FIG. 28 is a side view of an example of a lead or catheter based platform on which an echo Doppler sensor may be supported to position the sensor as discussed with respect to FIG. 4, 8, 10, 12, 14 or 16.

FIGS. 29 and 30 are side views of two versions of an example of a lead or catheter based platform on which an echo Doppler sensor may be supported to position the sensor as discussed with respect to FIG. 4, 8, 10, 12, 14 or 16.

FIGS. 31, 32 and 33 are side views of three versions of an example of a lead or catheter based platform on which an echo Doppler sensor may be supported to position the sensor as discussed with respect to FIG. 4, 8, 10, 12, 14 or 16.

FIGS. 34 and 35 are, respectively, side and isometric views of two versions of an example of a stent based platform on which an echo Doppler sensor may be supported to position the sensor as discussed with respect to FIG. 1, 4, 8, 10, 12, 14 or 16.

FIG. 36 is an isometric view of an example of a sensor for use with any of the devices discussed above with respect to FIGS. 19-35.

FIG. 37 is an isometric view of another example of a sensor for use with any of the devices discussed above with respect to FIGS. 19-35.

### DETAILED DESCRIPTION

Disclosed herein are medical devices 10 supporting one or more flow sensors 15 and configured to position the flow sensor in a desired location 20 within a patient's cardiovascular system such that the flow sensor will allow for hemodynamic monitoring and the identification of hemodynamic events. Sensor configurations and sensor placement locations within the patient's cardiovascular system are also disclosed herein.

The medical devices 10 may be implantable medical devices 10 such as, for example, stents, leads or lead-like devices that are implanted as part of a pacemaker system, implantable cardioverter defibrillator (“ICD”) system, or similar system. Alternatively, the medical devices 10 may be delivery devices 10 such as, for example, catheter, sheaths, or similar devices, or other devices 10 that are temporarily present within the patient during an implantation procedure, diagnostic procedure or other medical procedure.

The flow sensor 15 may be an echo Doppler or other flow sensor capable of being located in the arterial, venous or cardiac anatomy of a patient and configured to facilitate the measurement of blood flow velocity through an adjacent or neighboring target artery, vein, or cardiac structure of the patient. For example, in some embodiments, the sensor 15 is capable of being located outside the target vessel and sensing into the target vessel. For example, the sensor 15 can be located in venous vasculature and sense into an adjacent arterial vasculature to sense the aortic flow. Alternatively, the sensor 15 can be located adjacent the tricuspid valve annulus (e.g., adjacent the tricuspid valve annulus in the right atrium or right ventricle) or the mitral annulus (e.g., in the coronary sinus) and sense flow velocity across the tricuspid valve annulus or mitral annulus, respectively. The sensor 15 can also be located in the patient's vasculature or cardiac system to sense from the right side of the heart into the left side of the heart. Also, the sensor 15 can be located in the femoral vein and sense into the femoral artery or other similar situations where a vein is adjacent an artery, the sensor being located in the vein and the sensing being directed towards the adjacent artery.

Regardless of where the blood flow velocity measurements are taken, such measurements can be used as a direct measurement of hemodynamics. Identification of hemodynamic events can be used to determine which therapy is appropriate to provide to the patient.

The sensor 15 may be located on a variety of devices. For example, the device 10 may be a lead or lead-like device having a passive or active fixation tip, a helical lead body, an S-shaped lead body, a sleeve that slides over the lead and is sutured down a the site of venous access, a wire or other member that deflects outward against the wall of a vessel when tension or compression is applied to the wire or other member, a lead body that is deflected outward when a deflection wire of the lead is pulled or pushed, a biased and shaped deflection arm, or a slide arrangement with a portion that is deflected outward when a force is applied to, or released from, the slide arrangement. As another example, the device 10 may be a stent in the form of an expanding mesh or braid or an expandable laser cut tube made of, for example, Nitinol.

Implanting in a venous structure for sensing into an arterial structure can offer several advantages. For example, such a sensing arrangement can eliminate the need for anticoagulants. Sensing across the tricuspid annulus can be used to determine stroke volume. Sensing from the SVC into the SVC can be used to assess venous return from the upper portions of the body. Sensing from the subclavian vein into the subclavian artery can be used to identify sudden drops in or losses of flow to detect unstable ventricular tachycardia or ventricular fibrillation.

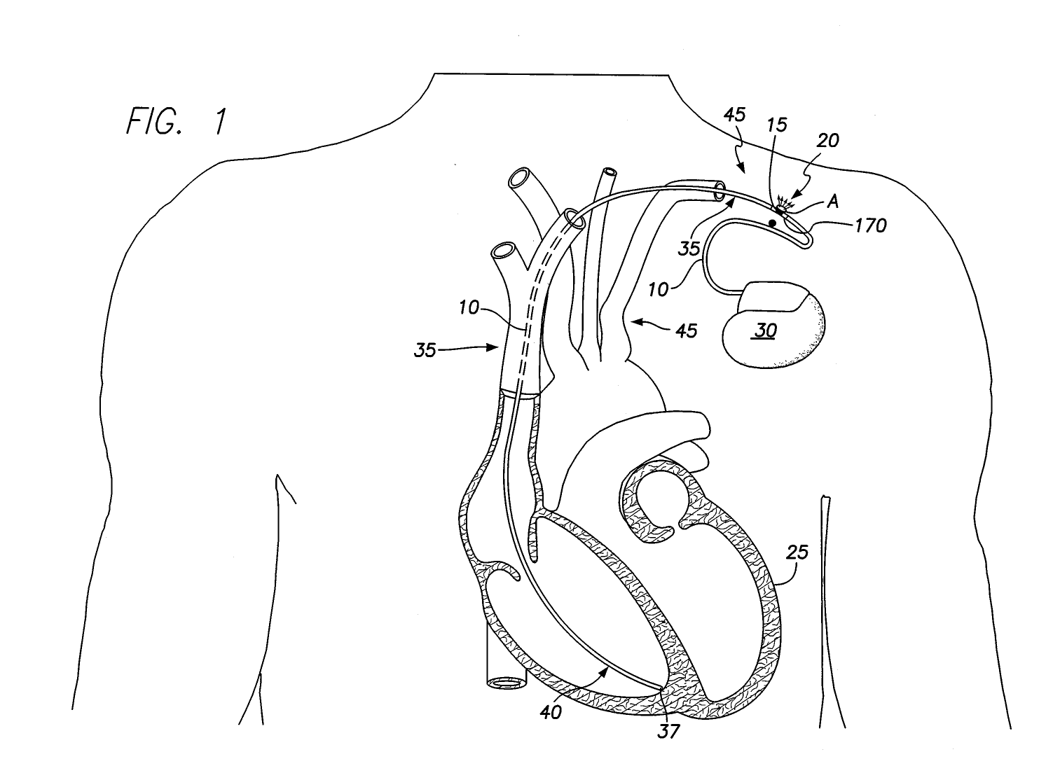

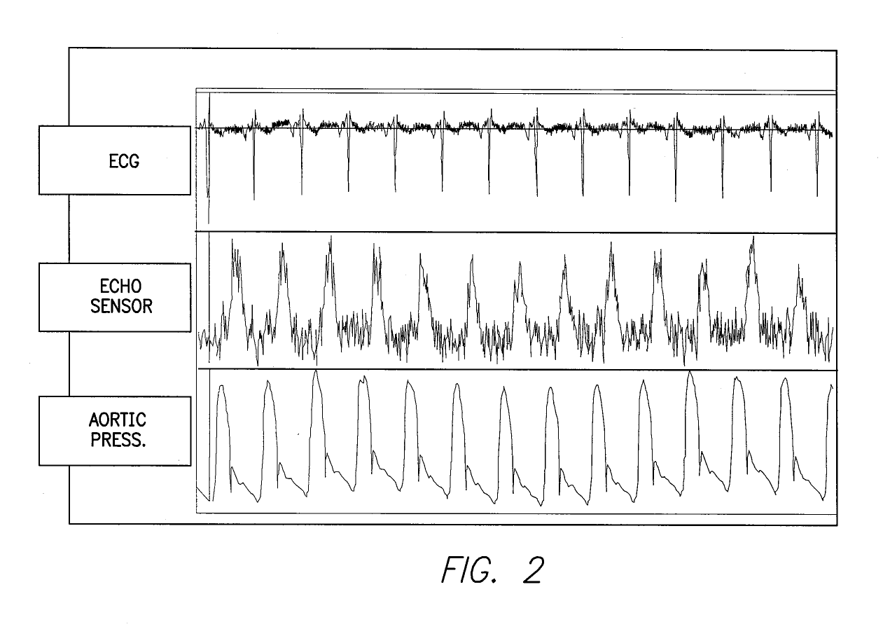

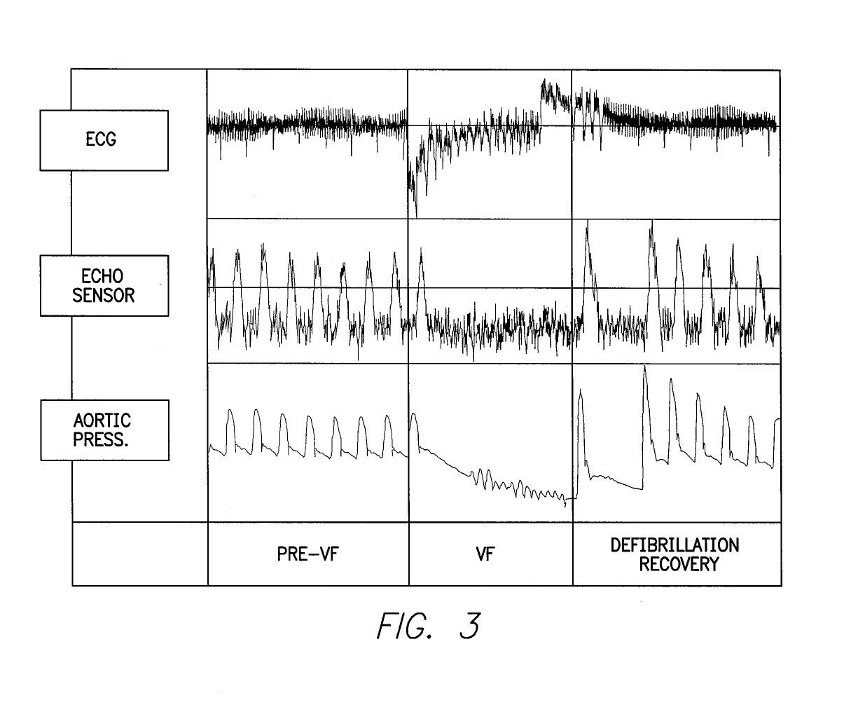

For a discussion of a first example sensor placement location 20 within the patient's cardiovascular system, reference is made to FIGS. 1-3. FIG. 1 is an anterior view of the patient's upper chest region with the heart 25 shown in partial cross section and illustrating the vasculature. FIG. 2 is a graphical representation of electrocardiogram (“ECG”), echo doppler sensor (“Echo Sensor”) and aortic pressure (“Aortic Press.”) data taken when the patient's heart is functioning normally and a flow sensor is positioned at the location 20 discussed with respect to FIG. 1. FIG. 3 is the same type of information taken when the patient's heart is undergoing ventricular fibrillation (“VF”) and recovery therefrom, the flow sensor being positioned at the location 20 discussed with respect to FIG. 1.

As shown in FIG. 1, in one embodiment, an implantable pulse generator 30 such as, for example, a pacemaker or implantable cardioverter defibrillator (“ICD”) is implanted in the patient's upper chest region near the patient's clavicle. An implantable medical lead 10 extends from the pulse generator 30 into the patient's subclavian vein 35 and into the patient's heart 25 such that the lead distal end 37 is located in, for example, the right ventricle 40 or another location within the patient's heart or vasculature. The flow sensor 15 is supported on or in the lead and, in one embodiment, is positioned in the subclavian vein in a location 20 wherein the subclavian vein is generally immediately adjacent, or peripheral, to the subclavian artery 45 and the flow sensor sensing projects towards the artery, as indicated by arrows A. As can be understood from FIG. 2, the flow sensor 15 positioned in the subclavian vein 35 as discussed with respect to FIG. 1 can be used to obtain aortic pressure data. As illustrated in FIG. 3, the aortic pressure data obtained via the flow sensor 15 at the location 20 in FIG. 1 can be used to sense VF and defibrillation recovery from VF.

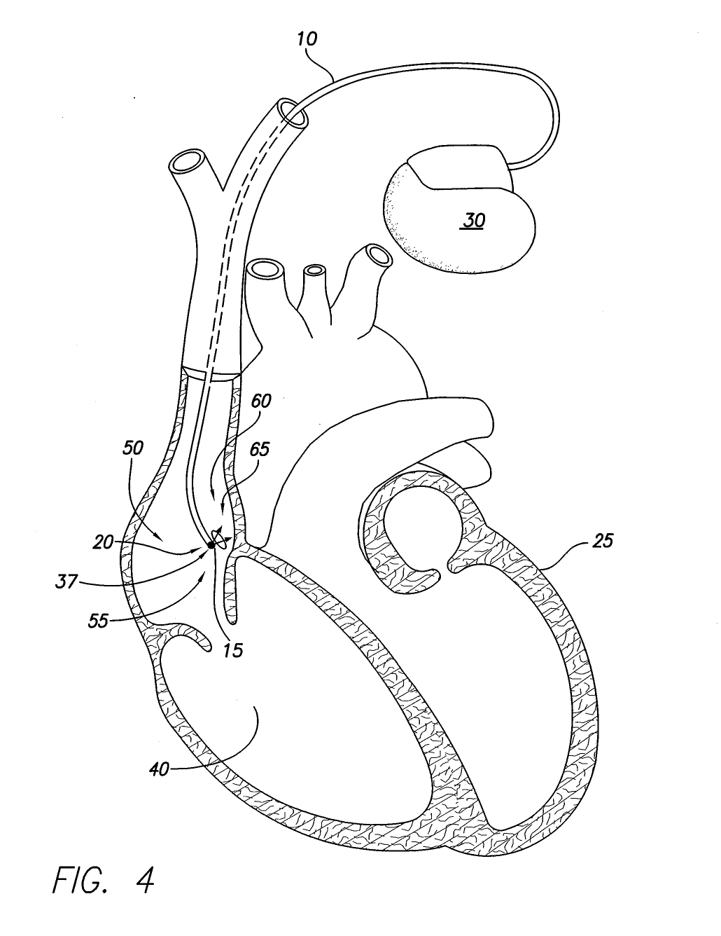

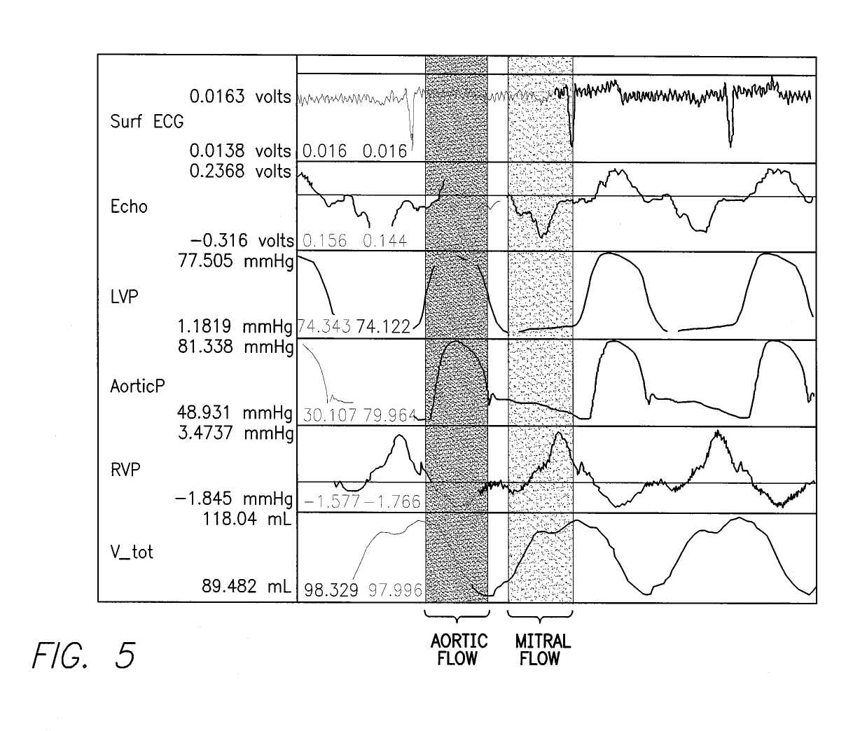

For a discussion of a second example sensor placement location 20 within the patient's cardiovascular system, reference is made to FIGS. 4-5. FIG. 4 is an anterior view of the heart 25 shown in partial cross section. FIG. 5 is a graphical representation of surface electrocardiogram (“Surf ECG”), echo doppler (“Echo”), left ventricular pressure (“LVP”), aortic pressure (“AorticP”), right ventricular pressure (“RVP”), and Left Ventricular Volume (“Vtot”) versus Aortic Flow and Mitral Flow when the patient's heart is functioning normally and a flow sensor 15 is positioned at the location 20 discussed with respect to FIG. 4.

As shown in FIG. 4, in one embodiment, an implantable medical lead 10 extends from a pulse generator 30 into the patient's vasculature (e.g., subclavian vein 35) and into the patient's heart 25 such that the lead distal end 37 is located in, for example, the right atrium 50 near the coronary sinus ostium 55. The flow sensor 15, which may be supported on or in the lead 10 near the lead distal end 37, is located in the right atrium 50 near the coronary sinus ostium 55 and generally against the right atrium wall 60 facing the aortic root 65. The flow sensor sensing projects towards the aortic root 65, as indicated by arrows B. As can be understood from FIG. 5, the flow sensor 15 positioned in the right atrium 50 near the coronary sinus ostium 55 and sensing towards the aortic root 65 as discussed with respect to FIG. 4 can be used to obtain flow velocity at the aortic root 65.

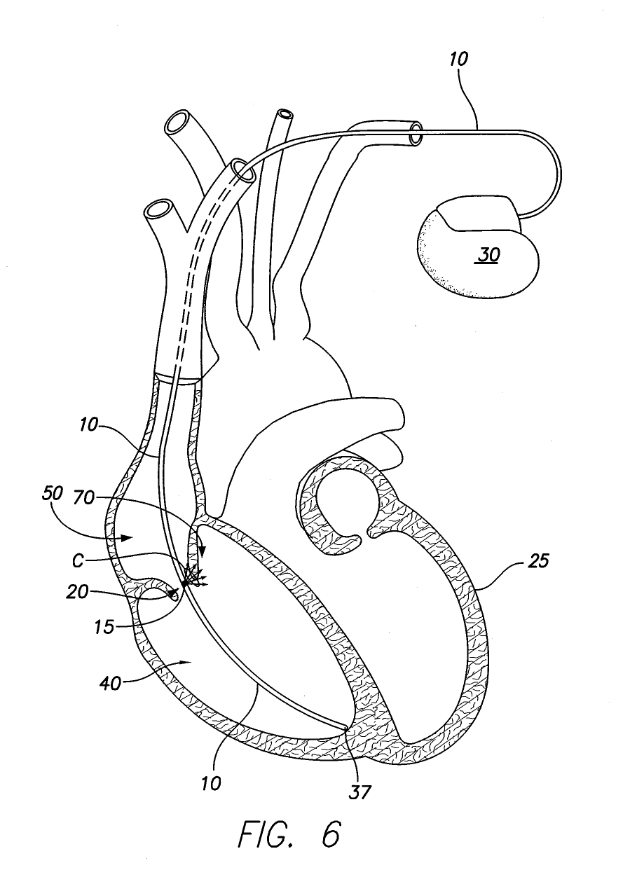

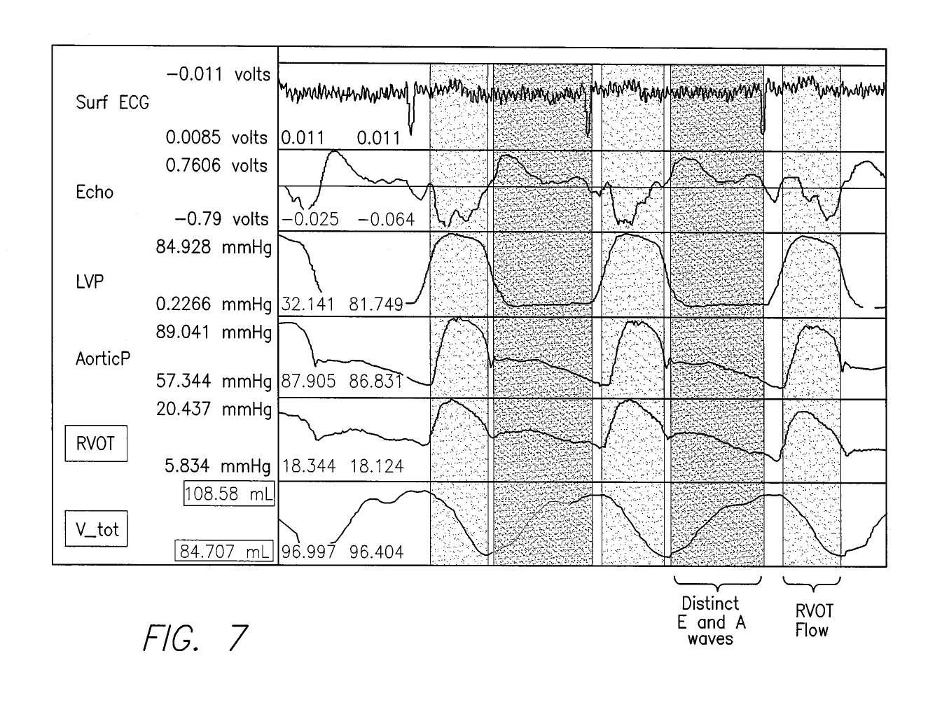

For a discussion of a third example sensor placement location 20 within the patient's cardiovascular system, reference is made to FIGS. 6-7. FIG. 6 is an anterior view of the heart 25 shown in partial cross section. FIG. 7 is a graphical representation of surface electrocardiogram (“Surf ECG”), echo doppler (“Echo”), left ventricular pressure (“LVP”), aortic pressure (“AorticP”), right ventricular outflow tract pressure (“RVOT”), and Left Ventricular Volume (“Vtot”) versus Distinct E and A waves and RVOT Flow when the patient's heart is functioning normally and a flow sensor 15 is positioned at the location 20 discussed with respect to FIG. 6.

As shown in FIG. 6, in one embodiment, an implantable medical lead 10 extends from a pulse generator 30 into the patient's vasculature (e.g., subclavian vein 35) and into the patient's heart 25 such that the lead distal end 37 is located in, for example, the right ventricle 40, extending through the tricuspid annulus 70. The flow sensor 15, which may be supported on or in the lead 10, is located in the tricuspid annulus 70. The flow sensor sensing projects towards the tricuspid annulus 70, as indicated by arrows C. As can be understood from FIG. 7, the flow sensor 15 positioned in and sensing towards the tricuspid annulus 70 as discussed with respect to FIG. 6 can be used to obtain flow velocity across the tricuspid annulus.

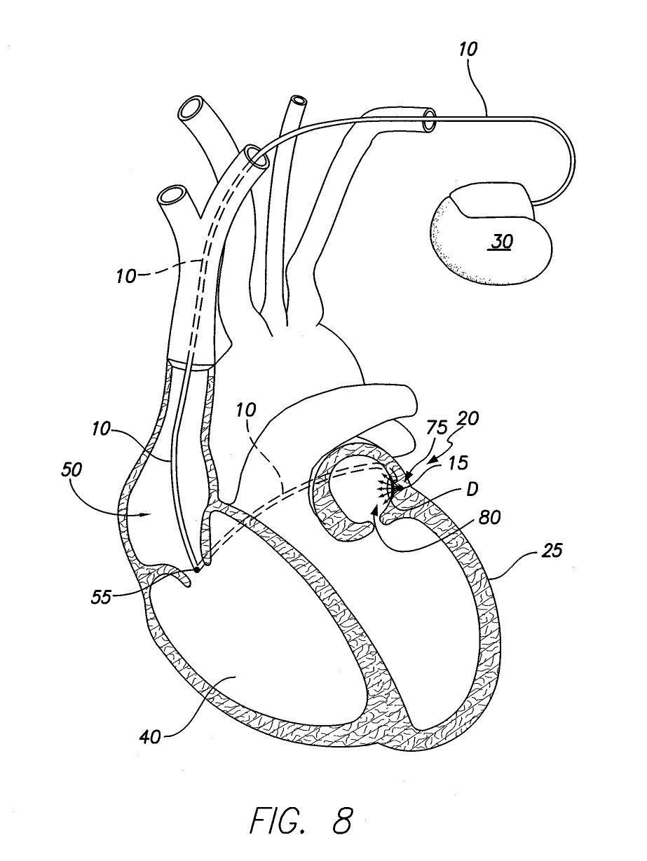

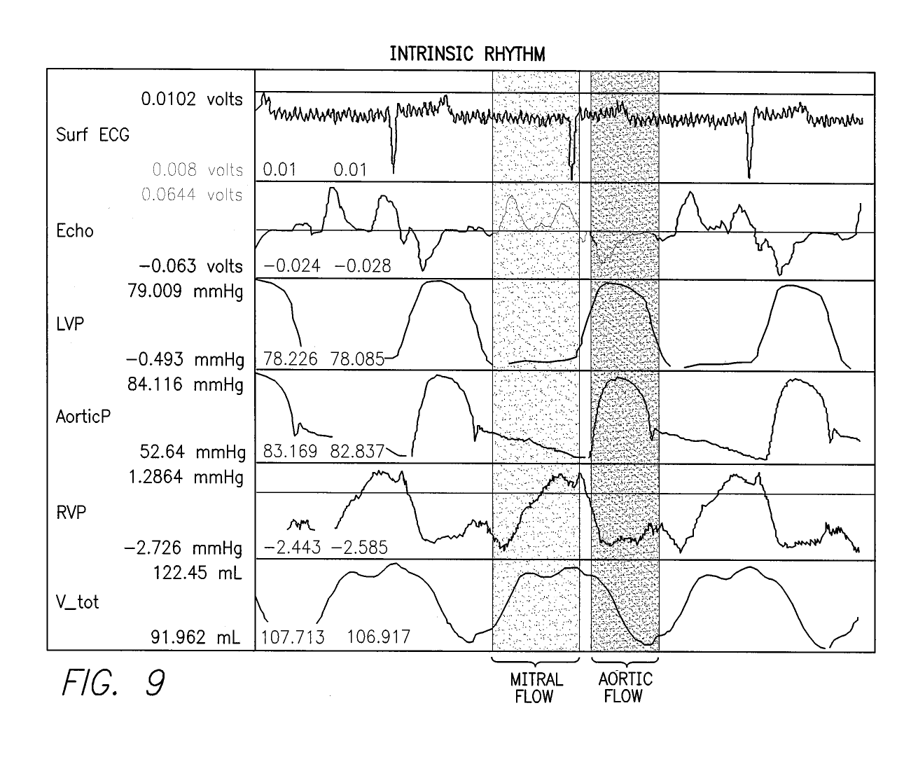

For a discussion of a fourth example sensor placement location 20 within the patient's cardiovascular system, reference is made to FIGS. 8-9. FIG. 8 is an anterior view of the heart 25 shown in partial cross section. FIG. 9 is a graphical representation for intrinsic rhythm of surface electrocardiogram (“Surf ECG”), echo doppler (“Echo”), left ventricular pressure (“LVP”), aortic pressure (“AorticP”), right ventricular pressure (“RVP”), and Left Ventricular Volume (“Vtot”) versus Aortic Flow and Mitral Flow when the patient's heart is functioning normally and a flow sensor 15 is positioned at the location 20 discussed with respect to FIG. 8.

As shown in FIG. 8, in one embodiment, an implantable medical lead 10 extends from a pulse generator 30 into the patient's vasculature (e.g., subclavian vein 35) and into the patient's heart 25 such that the lead extends through the right atrium 50 and coronary sinus ostium 55 into the coronary sinus 75. The flow sensor 15, which may be supported on or in the lead 10 near the lead distal end 37, is located in the coronary sinus 75 and generally against the left wall of the heart facing the mitral annulus 80. The flow sensor sensing projects towards the mitral annulus 80, as indicated by arrows D. As can be understood from FIG. 9, the flow sensor 15 positioned in the coronary sinus 75 and sensing towards the mitral annulus 80 as discussed with respect to FIG. 8 can be used to obtain flow velocity across the mitral annulus and LV outflow tract. Forward and reverse flow across the mitral annulus can be measured. The associated flow velocity can be used to determine cardiac output, ischemia, optimal atrial to ventricular timing, left ventricular contractility, and distinguish between SVT and VT.

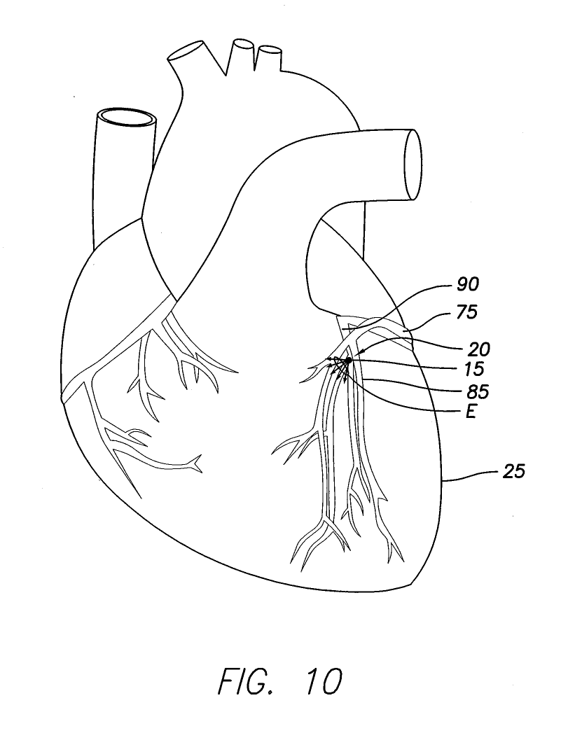

For a discussion of a fifth example sensor placement location 20 within the patient's cardiovascular system, reference is made to FIGS. 10-11. FIG. 10 is an anterior view of the heart 25. FIG. 11 is a graphical representation of surface electrocardiogram (“Surf ECG”), echo doppler (“Echo”), left ventricular pressure (“LVP”), aortic pressure (“AorticP”), right atrial pressure (“RA P”), and Left Ventricular Volume (“Vtot”) when the patient's heart is functioning normally and a flow sensor 15 is positioned at the location 20 discussed with respect to FIG. 10.

In a manner similar to that discussed with respect to FIG. 8, in one embodiment, an implantable medical lead 10 extends from a pulse generator 30 into the patient's vasculature (e.g., subclavian vein 35) and into the patient's heart 25 such that the lead extends through the right atrium 50 and coronary sinus ostium 55 into the coronary sinus 75. The lead 10 further extends into the great coronary vein 85 from the coronary sinus 75, as can be understood from FIG. 10. The flow sensor 15, which may be supported on or in the lead 10 near the lead distal end 37, is located in the great coronary vein 85 and faces the left anterior descending artery 90. The flow sensor sensing projects towards the left anterior descending artery 90, as indicated by arrows E. As can be understood from FIG. 11, the flow sensor 15 positioned in the great coronary vein 85 and sensing towards the left anterior descending artery 90 as discussed with respect to FIG. 10 can be used to obtain flow velocity in the left anterior descending artery 90.

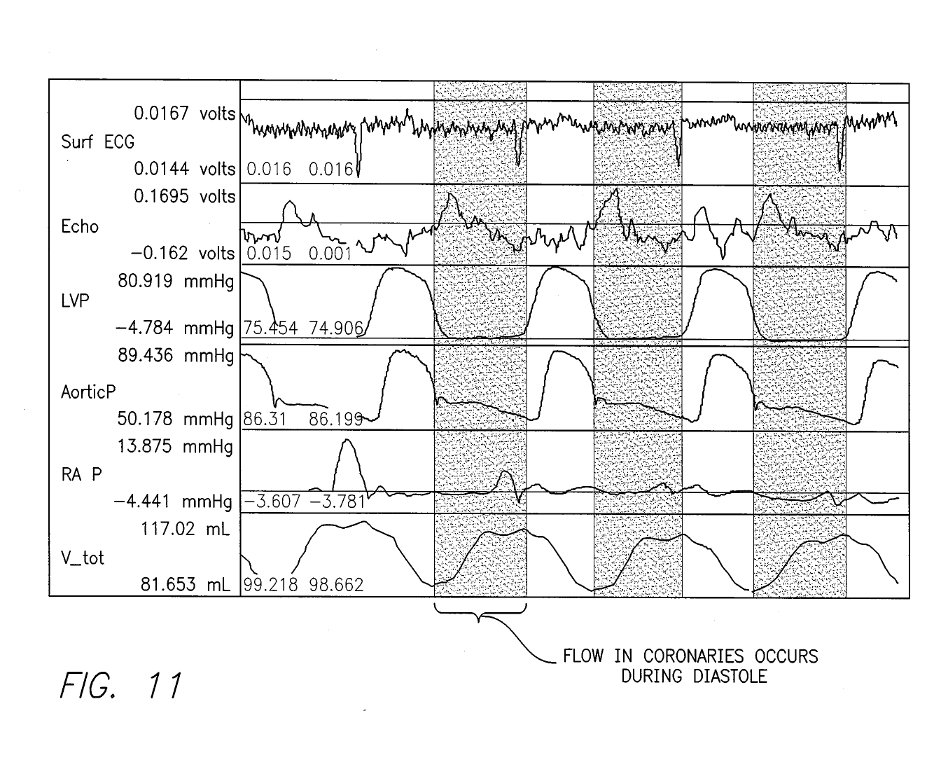

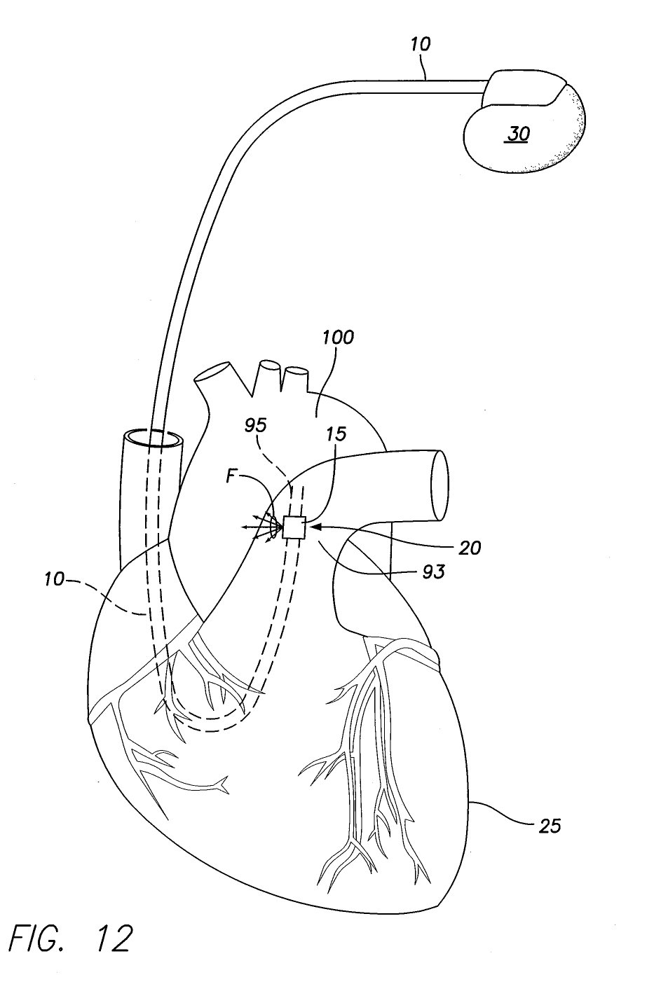

For a discussion of a sixth example sensor placement location 20 within the patient's cardiovascular system, reference is made to FIGS. 12-13. FIG. 12 is an anterior view of the heart 25. FIG. 13 is a graphical representation for intrinsic rhythm of electrocardiogram (“ECG”), echo doppler (“Sensor”), left ventricular pressure (“LV P”), aortic pressure (“Aortic P”), Left Ventricular Volume (“LV VOL”), and Echo Sound Recording (“EchoSnd”) when the patient's heart is functioning normally and a flow sensor 15 is positioned at the location 20 discussed with respect to FIG. 12.

As can be understood from FIG. 12, in one embodiment, a catheter, sheath or other tubular body 10 extends into the right ventricular outflow tract (“RVOT”) 93 such that a flow sensor 15, which may be supported on or in the catheter 10 near the distal end 95 of the catheter, is located in the RVOT and faces the aorta 100. The flow sensor sensing projects towards the aorta 100, as indicated by arrows F. As can be understood from FIG. 13, the flow sensor 15 positioned in the RVOT 93 and sensing towards the aorta 100 as discussed with respect to FIG. 12 can be used to obtain flow velocity in the aorta 100.

For a discussion of a seventh example sensor placement location 20 within the patient's cardiovascular system, reference is made to FIGS. 14-15. FIG. 14 is an anterior view of the heart 25. FIG. 15 is a graphical representation for intrinsic rhythm of electrocardiogram (“ECG”), echo doppler (“Sensor”), left ventricular pressure (“LV P”), aortic pressure (“Aortic P”), Left Ventricular Volume (“LV VOL”), and Echo Sound Recording (“EchoSnd”) when the patient's heart is functioning normally and a flow sensor 15 is positioned at the location 20 discussed with respect to FIG. 12.

As can be understood from FIG. 14, in one embodiment, a catheter, sheath or other tubular body 10 extends into the right atrial appendage (“RA appendage”) 105 such that a flow sensor 15, which may be supported on or in the catheter 10 near the distal end 95 of the catheter, is located in the RA appendage and faces the aorta 100. The flow sensor sensing projects towards the aorta 100, as indicated by arrows G. As can be understood from FIG. 15, the flow sensor 15 positioned in the RA appendage 105 and sensing towards the aorta 100 as discussed with respect to FIG. 14 can be used to obtain flow velocity in the aorta 100.

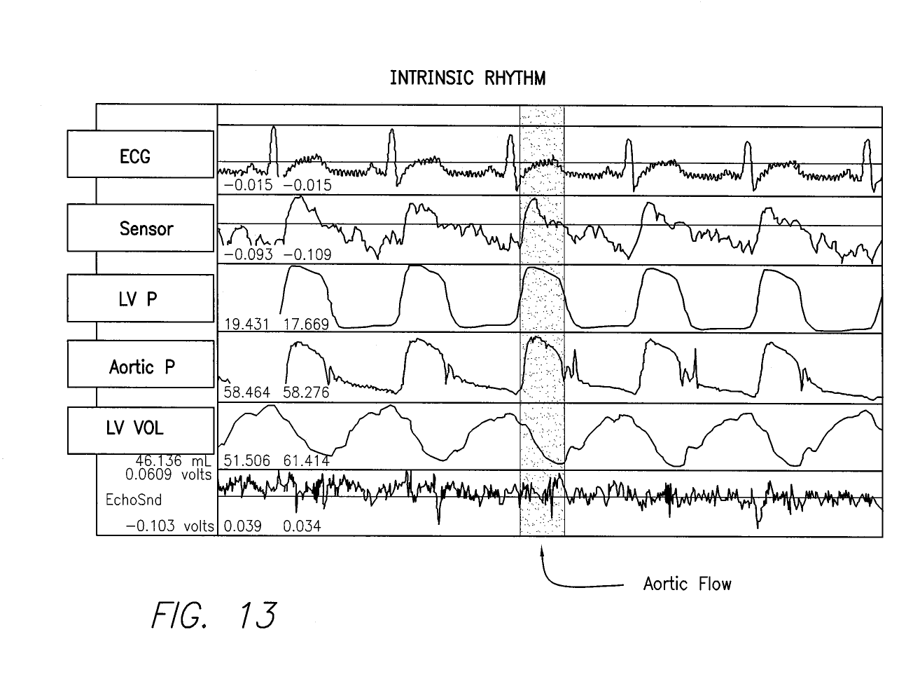

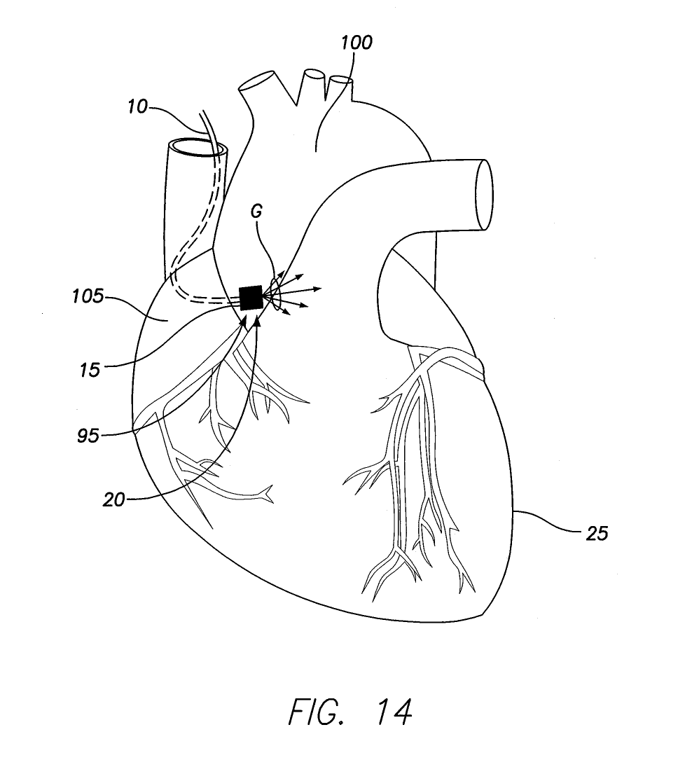

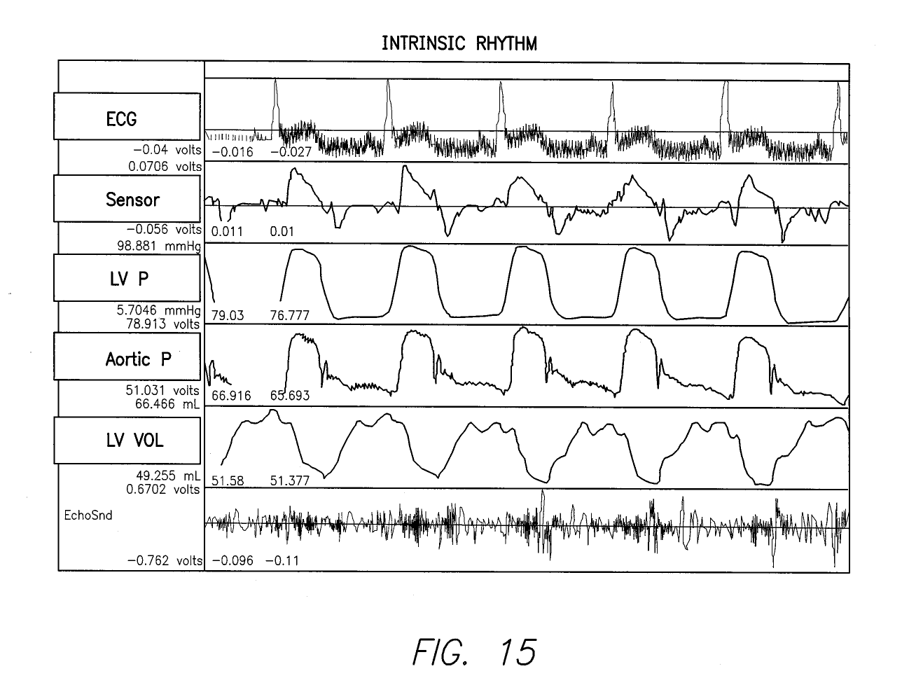

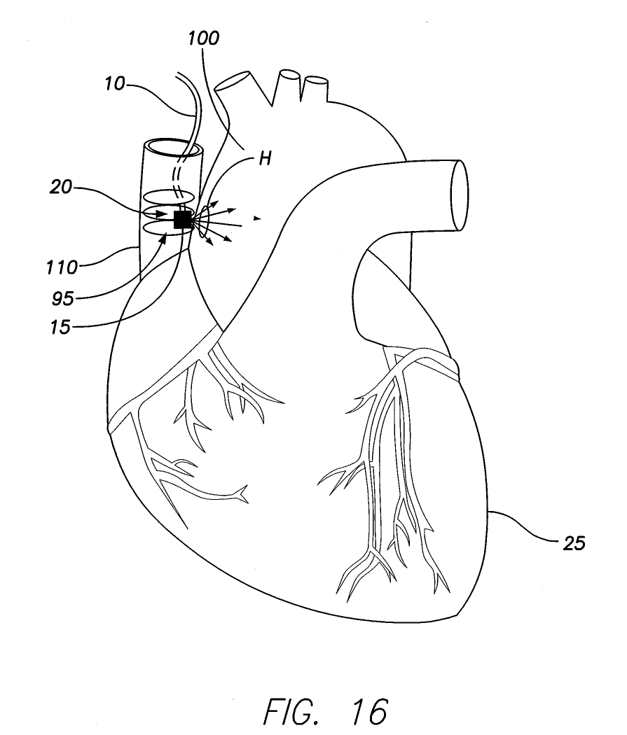

For a discussion of an eighth example sensor placement location 20 within the patient's cardiovascular system, reference is made to FIGS. 16-18. FIG. 16 is an anterior view of the heart 25. FIG. 17 is a graphical representation for intrinsic rhythm of electrocardiogram (“ECG”), echo Doppler (“Sensor”), left ventricular pressure (“LV P”), aortic pressure (“Aortic P”), and Left Ventricular Volume (“LV VOL”) when the patient's heart is functioning normally and a flow sensor 15 is positioned at the location 20 discussed with respect to FIG. 16. FIG. 18 is a graphical representation for ventricular tachycardia (“VT”) to ventricular fibrillation (“VF”) transition of electrocardiogram (“ECG”), echo doppler (“Sensor”), left ventricular pressure (“LV P”), aortic pressure (“Aortic P”), Left Ventricular Volume (“LV VOL”), and Echo Sound Recording (“EchoSnd”) when the patient's heart is functioning normally and a flow sensor 15 is positioned at the location 20 discussed with respect to FIG. 16.

As can be understood from FIG. 16, in one embodiment, a lead 10 extends into the superior vena cava (“SVC”) 110 such that a flow sensor 15, which may be supported on or in the lead 10 near the distal end 95 of the lead, is located in the SVC and faces the aorta 100. The flow sensor sensing projects towards the aorta 100, as indicated by arrows H. As can be understood from FIGS. 17-18, the flow sensor 15 positioned in the SVC 110 and sensing towards the aorta 100 as discussed with respect to FIG. 16 can be used to obtain flow velocity in the aorta 100.

As can be understood from the preceding discussion regarding FIGS. 1-18, the sensor 15 may be placed in a variety of locations within the patient's vasculature and cardiac structure to sense flow velocity in a variety of other locations within the patient's vasculature and cardiac structure. Examples of such sensor placement and sensing location combinations include, but are not limited to, the following: placement of the sensor in the SVC for sensing blood flow in the ascending aorta; placement of the sensor in the CS for sensing blood flow across the mitral annulus and out the left ventricular outflow; placement of the sensor in the right atrial appendage for sensing blood flow in the aorta; placement of the sensor in the right ventricular outflow tract for sensing blood flow in the aorta; placement of the sensor in the subclavian vein for sensing blood flow in the subclavian artery; placement of the sensor in the tricuspid valve annulus to sense blood flow across the tricuspid valve or tricuspid valve annulus; placement of the sensor in the great cardiac vein to sense blood flow in the left anterior descending artery; placement of the sensor in the right atrium near the coronary sinus ostium to sense blood flow in the aortic root; and placement of the sensor in the superior vena cava to sense blood flow in the superior vena cava or ascending aorta.

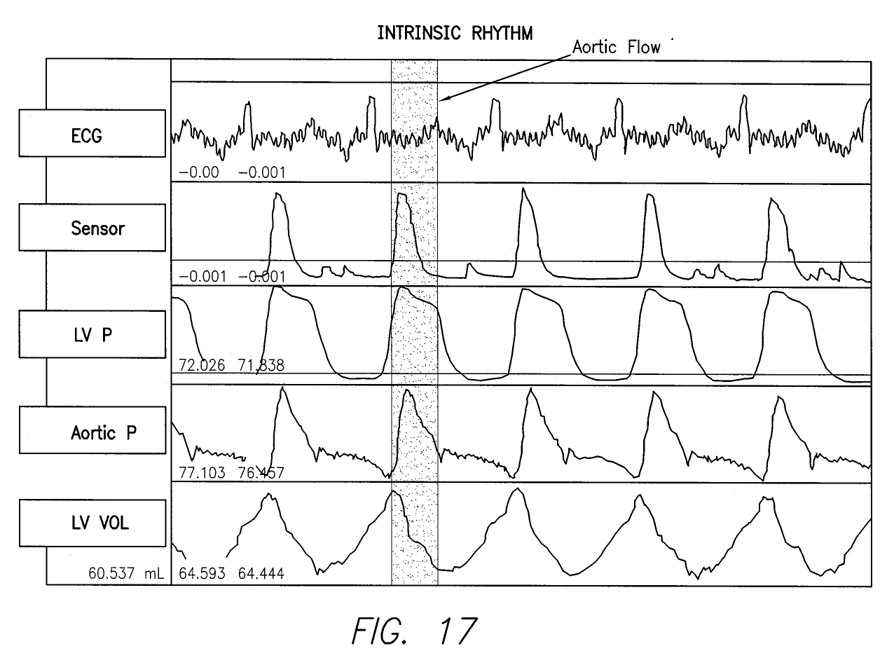

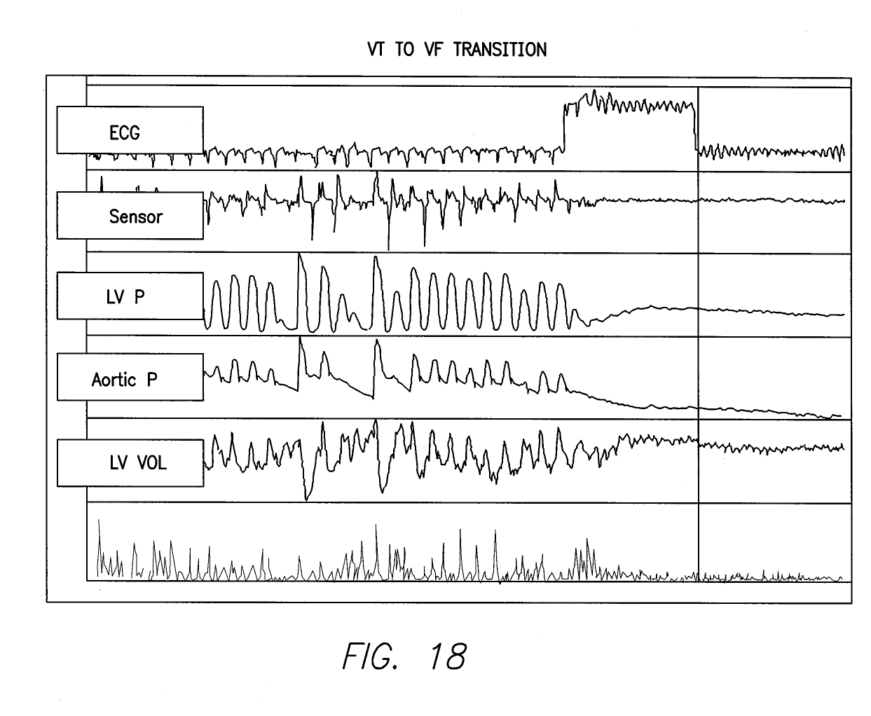

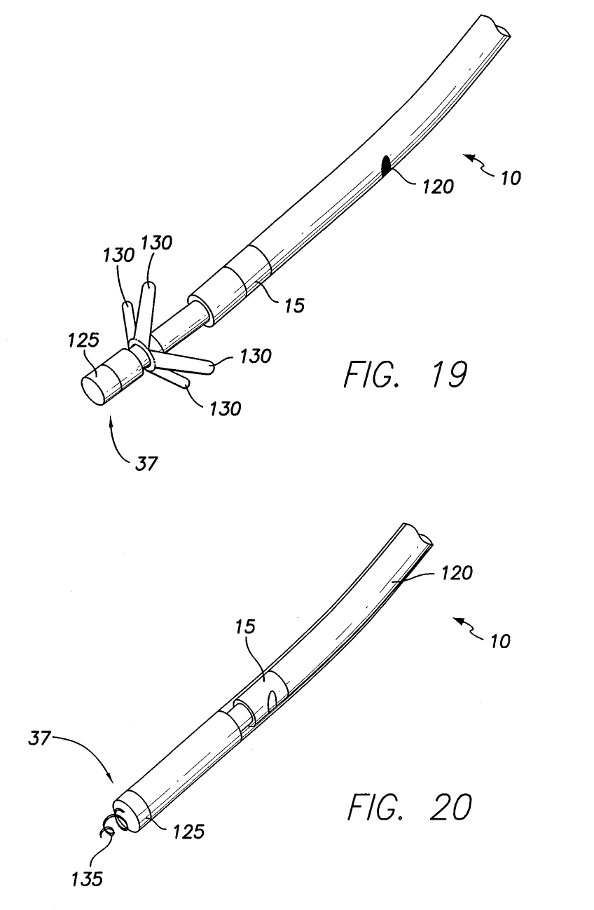

For a discussion of a first example of a lead based platform on which an echo doppler sensor 15 may be supported to position the sensor 15 as discussed above with respect to FIG. 4, for example, reference is now made to FIGS. 19 and 20, which are, respectively, isometric views of a lead distal end 37 having a passive fixation configuration and a lead distal end 37 having an active fixation configuration. As shown in FIGS. 19 and 20, each of the leads 10 includes a tubular body 120 and an echo Doppler sensor 15 supported on or in the lead body. Each lead may also further include electrodes 125 for pacing, sensing and/or shocking, as is known in the art. As indicated in FIG. 19, the lead distal end may include pliable arms or tines 130 for passively fixing the lead distal end at a desired implantation site. Alternatively, as illustrated in FIG. 20, the lead distal end may include a helical anchor 135 for actively fixing the lead distal end at a desired implantation site. In either case, the leads of FIGS. 19 and 20 will have the sensor 15 oriented on the lead body such that when the lead distal end is implanted near the coronary sinus ostium 55 as indicated in FIG. 4, the sensor 15 will be sensing towards the aortic root 65.

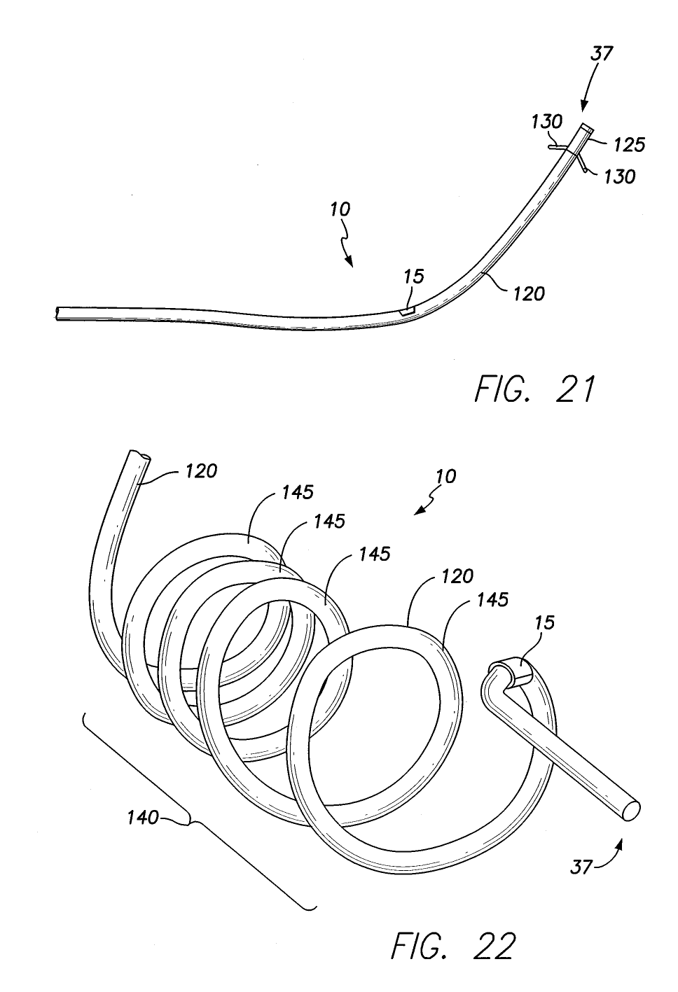

As shown in FIG. 21, which is a side view of a second example of a lead based platform on which an echo doppler sensor 15 may be supported to position the sensor 15 as discussed above with respect to FIG. 6, for example, the lead distal end 37 may be configured similar to either of the lead distal ends depicted in FIG. 19 or 20 to have either a passive or active fixation configuration. However, the lead 10 of FIG. 21 will have its echo doppler sensor 15 located more proximally such that when the lead distal end is implanted near the apex of the right ventricle 40 as depicted in FIG. 6, the sensor 15 will be positioned in or near the tricuspid annulus 70 in such an orientation to allow the sensor 15 to sense flow velocity across the tricuspid annulus 70.

As shown in FIG. 22, which is an isometric view of a third example of a lead based platform on which an echo doppler sensor 15 may be supported to position the sensor 15 as discussed above with respect to FIG. 16, for example, the lead body 120 has a helical portion 140. Specifically, the helical portion 140 is formed of the lead body 120 wound in approximately three to six helical coils 145 having diameters sufficiently wide to cause the helical coils to abut against the walls of the SVC with sufficient force to maintain the sensor in position as indicated in FIG. 16. In other words, once the lead 10 is delivered to the SVC via a delivery tool such as, for example, a catheter, sheath, guidewire and/or stylet, and the delivery tool is removed from the lead in the SVC, the lead body biases into the helical form depicted in FIGS. 22 and 16. The echo Doppler sensor 15 is supported in or on the lead body such that when the helical portion 140 is implanted in the SVC as depicted in FIG. 16, the sensor 15 will be positioned in such an orientation to allow the sensor 15 to sense flow velocity in the aorta 100.

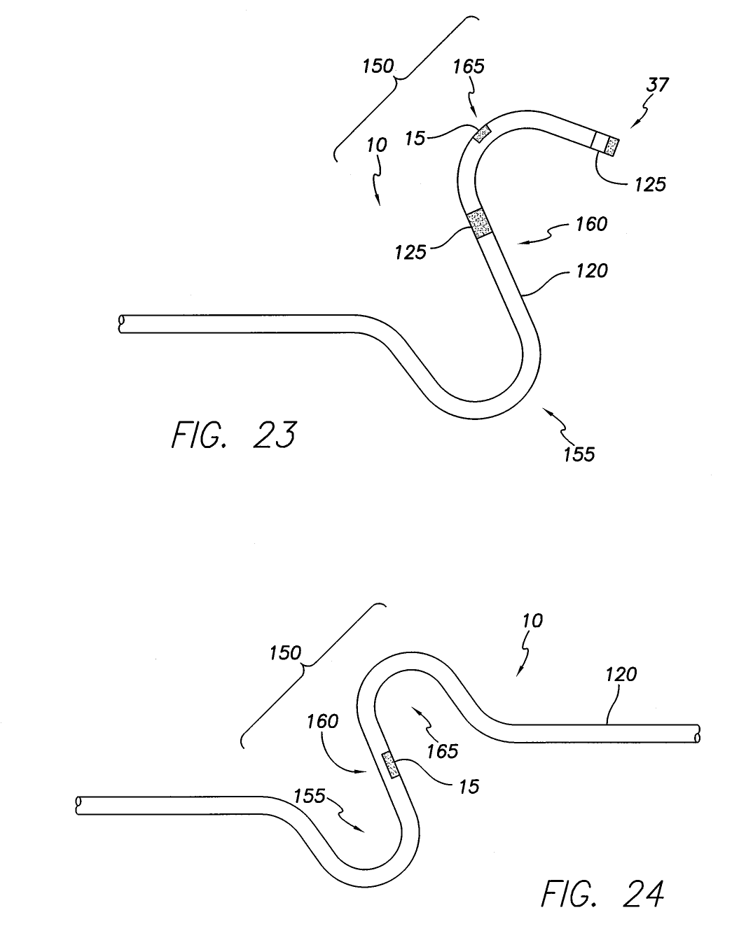

As shown in FIG. 23, which is a side view of a fourth example of a lead based platform on which an echo doppler sensor 15 may be supported to position the sensor 15 as discussed above with respect to FIG. 8 or 10, for example, the lead body 120 has a curved portion 150. Specifically, the curved portion 150 has a proximal curve 155 of approximately 180 degrees extending distally via a straight segment 160 into a distal curve 165 of approximately 135 degrees. The sensor 15 is located on or in the lead body near the midpoint of the distal curve 165. The curved portion 150 is configured to cause the proximal and distal curves to abut against the walls of the CS or great cardiac vein with sufficient force to maintain the lead in position as indicated in FIG. 8 or 10. In other words, once the lead 10 is delivered to the CS or great cardiac vein via a delivery tool such as, for example, a catheter, sheath, guidewire and/or stylet, and the delivery tool is removed from the lead in the CS or great cardiac vein, the lead body biases into the curved form depicted in FIG. 23. In one embodiment, the echo Doppler sensor 15 is supported in or on the lead body such that when the curved portion 150 is implanted in the CS 75 as depicted in FIG. 8, the sensor 15 will be positioned in such an orientation to allow the sensor 15 to sense flow velocity across the mitral annulus 80. In another embodiment, the echo doppler sensor 15 is supported in or on the lead body such that when the curved portion 150 is implanted in the great coronary vein 85 as depicted in FIG. 10, the sensor 15 will be positioned in such an orientation to allow the sensor 15 to sense flow velocity in the left anterior descending artery 90. In either embodiment, the lead body may also include electrodes 125 for pacing, sensing and/or shocking.

As shown in FIG. 24, which is a side view of a fifth example of a lead based platform on which an echo doppler sensor 15 may be supported to position the sensor 15 as discussed above with respect to FIG. 6 or 16, for example, the lead body 120 has a curved portion 150. Specifically, the curved portion 150 has a proximal curve 155 of approximately 180 degrees extending distally via a straight segment 160 into a distal curve 165 of approximately 180 degrees. The sensor 15 is located on or in the lead body near the midpoint of the straight segment 160. In one embodiment, the curved portion 150 is configured to cause the proximal and distal curves to abut against the walls 170 of the SVC 110, as indicated in FIG. 24, with sufficient force to maintain the lead in position as can be understood from FIG. 16. In other words, once the lead 10 is delivered to the SVC via a delivery tool such as, for example, a catheter, sheath, guidewire and/or stylet, and the delivery tool is removed from the lead in the SVC, the lead body biases into the curved form depicted in FIG. 24. In one embodiment, the echo Doppler sensor 15 is supported in or on the lead body such that when the curved portion 150 is implanted in the SVC, the sensor 15 will be positioned in such an orientation to allow the sensor 15 to sense flow velocity in the SVC.

As can be understood from FIGS. 24 and 6, in one embodiment, the echo Doppler sensor 15 is supported in or on the lead body such that when the curved portion 150 is implanted in or near the tricuspid annulus 70, the sensor is oriented to point in the direction of the flow through the tricuspid annulus. As a result, the sensor 15 will be positioned in such an orientation to allow the sensor 15 to sense flow velocity across the tricuspid annulus 70.

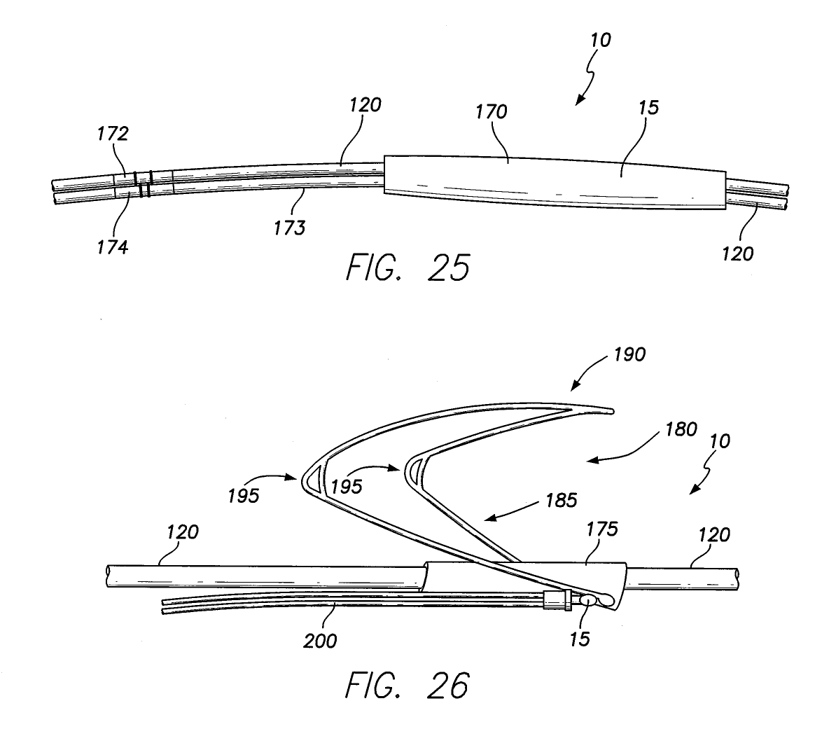

As shown in FIG. 25, which is a side view of a sixth example of a lead based platform on which an echo doppler sensor 15 may be supported to position the sensor 15 as discussed above with respect to FIG. 1, for example, the lead body 120 includes a suture sleeve 170 on which the sensor 15 is supported. Once the lead 10 is implanted, for example, as depicted in FIG. 1, the suture sleeve 170 may be sutured in place in the subclavian vein or at the access into the subclavian vein such the sensor 15 is positioned in such an orientation to allow the sensor 15 to sense flow velocity in the adjacent subclavian artery 45.

As illustrated in FIG. 25, in one embodiment, the lead body 120 proximal of the sheath 170 carries electrical conductors from the lead electrodes, sensor and shock coils to a lead connector end 172 for coupling to the pulse generator 30. Another lead body portion 173 proximal of the sheath 170 carries electrical conductors from the doppler sensor 15 in the sheath 170 to another lead connector end 174 for coupling to the pulse generator 30 or other monitoring designed to monitor fluid velocity via the sensor 15.

As shown in FIG. 26, which is a side view of a seventh example of a lead or catheter based platform on which an echo doppler sensor 15 may be supported to position the sensor 15 as discussed above with respect to FIG. 4, 8, 10, 12, 14 or 16, for example, the lead or catheter body 120 includes a sleeve 175 on which the sensor 15 is supported. The sleeve extends about the tubular body 120, and a biased arm 180 extends from the sleeve. The biased arm may be in the form of a folded loop having a lower portion 185 attached to the sleeve and an upper portion 190 joining the lower portion at a hinge like bend 195. The biased arm 180 is configured to cause the sleeve and arm to abut against the walls of the surrounding cardiac structure (e.g., SVC, right atrium, RVOT, CS, great cardiac vein, or etc.) with sufficient force to maintain the device 10 in position as indicated in FIG. 4, 8, 10, 12, 14 or 16. In other words, once the device 10 is delivered to the volume of a desired cardiac structure via a delivery tool such as, for example, a catheter or sheath, and the delivery tool is removed from about the device 10 in the volume of the desired cardiac structure, the arm 180 biases into the configuration depicted in FIG. 26. In one embodiment, the echo doppler sensor 15 is supported in or on the sleeve 175 such that when the sleeve is implanted in the volume of the desired cardiac structure, the sensor 15 will be positioned in such an orientation to allow the sensor 15 to sense flow velocity across the a targeted flow region, such as, for example, the aortic root 65 (see FIG. 4), the mitral annulus 80 (see FIG. 8), the left anterior descending artery 90 (see FIG. 10), or the aorta 100 (see FIG. 12, 14 or 16).

In one embodiment, the device 10 is a lead and the sleeve 175 with its sensor 15 may be permanently implanted at the desired monitoring location. In other embodiments, the device 10 is a catheter or sheath that carries the sleeve with its sensor 15 to the desired monitoring location to permanently implant the sleeve 175 and sensor 15 at the implantation site (the catheter or sleeve being withdrawn from the sleeve, leaving the sensor equipped sleeve behind). Alternatively, the catheter or sheath is used to locate the sensor equipped sleeve at the desired monitoring location, the catheter or sleeve being withdrawn from the monitoring location when the monitoring is completed, the sleeve and sensor being withdrawn by the withdrawing of the catheter or sleeve.

As indicated in FIG. 26, electrical conductors 200 extend proximally from the sensor 15. Depending on the embodiment, the conductors 200 may be solid core wires, multi-filar cables, etc. The conductors 200 extend through and out of the patient to electrically couple to an echo Doppler monitoring and control apparatus for monitoring, controlling and displaying the signals received by the sensor 15. Of course, although conductors 200 are not illustrated in the other embodiments depicted in FIGS. 19-25 and 27-35, conductors 200 do extend along the lead or catheter tubular body 120 from the sensor 15 to the associated fluid velocity monitoring system (which may be located in the pulse generator 30).

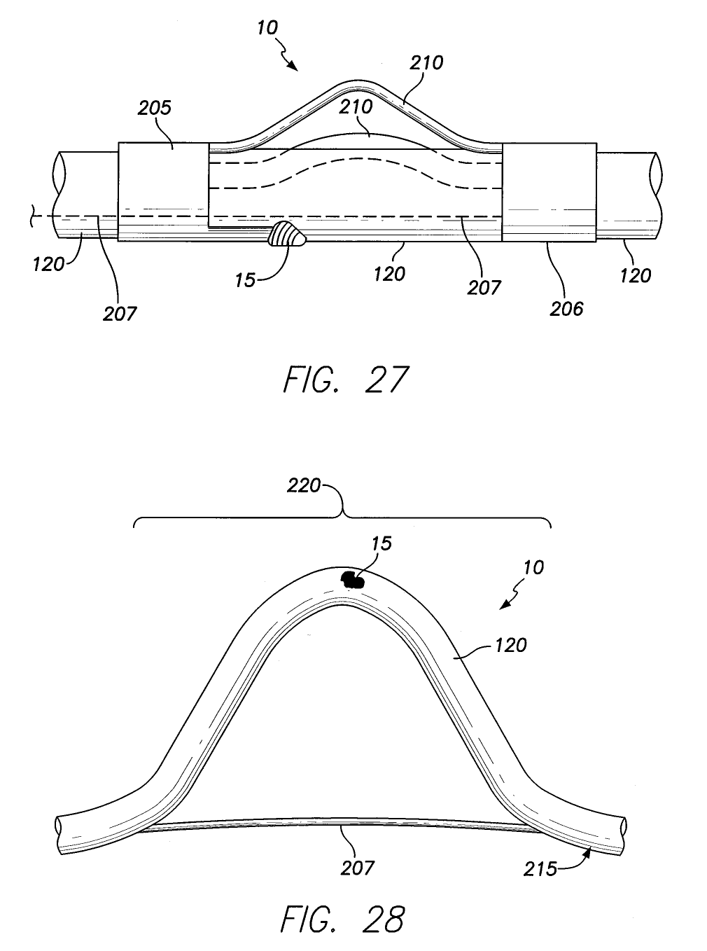

As indicated in FIG. 27, which is a side view of an eighth example of a lead or catheter based platform 10 on which an echo doppler sensor 15 may be supported to position the sensor 15 as discussed above with respect to FIG. 4, 8, 10, 12, 14 or 16, for example, the lead or catheter body 120 includes a sleeve, collar, ring or other structure 205 on which the sensor 15 is supported. In one embodiment, first and second rings 205, 206 extend about the tubular body 120 and are spaced apart from each other proximal-distal. The distal ring 206 is slidable along the tubular body and the proximal ring 205 is fixed to the tubular body 120. In one embodiment, one or more biased arms 210 extend between the rings 205, 206 from the sleeve and bias into a configuration wherein a midpoint of each arm 210 is spaced apart from the tubular body 120 when not restrained by a delivery tool such as, for example, a sheath or catheter, the distal ring sliding along the tubular body towards the proximal ring when the arms 210 are freed to bias outwards. In another embodiment, a deflection wire 207 extends proximally from the distal ring 206 and the wire 207 can be pulled proximally to cause the distal ring 206 to slide towards the proximal ring 205, causing the arms 210 to deflect outward.

The one or more arms 210 are configured to cause the tubular body 120 and arms 210 to abut against the walls of the surrounding cardiac structure (e.g., SVC, right atrium, RVOT, CS, great cardiac vein, or etc.) with sufficient force to maintain the device 10 in position as indicated in FIGS. 4, 8, 10, 12, 14 or 16. Thus, in one embodiment, once the device 10 is delivered to the volume of a desired cardiac structure via a confining delivery tool such as, for example, a catheter or sheath, and the confining delivery tool is removed from about the device 10 in the volume of the desired cardiac structure, the freed arms 210 bias into the configuration depicted in FIG. 27. Alternatively, in one embodiment, once the device 10 is delivered to the volume of a desired cardiac structure via a delivery tool such as, for example, a catheter, sheath, guidewire or stylet, and the pull wire 207 is pulled to cause the distal slide ring 206 to move towards the proximal fixed ring 205, causing the arms 210 to deflect away from the tubular body 120 in the volume of the desired cardiac structure, the deflected arms 210 assume the configuration depicted in FIG. 27.

In one embodiment, the echo doppler sensor 15 is supported on or off of the assembly of rings 205, 206 such that when the assembly of rings is implanted in the volume of the desired cardiac structure, the sensor 15 will be positioned in such an orientation to allow the sensor 15 to sense flow velocity across the a targeted flow region, such as, for example, the aortic root 65 (see FIG. 4), the mitral annulus 80 (see FIG. 8), the left anterior descending artery 90 (see FIG. 10), or the aorta 100 (see FIG. 12, 14 or 16).

In one embodiment, the device 10 is a lead and the assembly of rings 205, 206 with its sensor 15 may be permanently implanted at the desired monitoring location. In other embodiments, the device 10 is a catheter or sheath that carries the assembly of rings 205, 206 with its sensor 15 to the desired monitoring location to permanently implant the assembly of rings 205, 206 and sensor 15 at the implantation site (the catheter or sleeve being withdrawn from the assembly of rings 205, 206, leaving the sensor equipped assembly behind). Alternatively, the catheter or sheath is used to locate the sensor equipped assembly at the desired monitoring location, the catheter or sleeve being withdrawn from the monitoring location when the monitoring is completed, the assembly of rings and sensor being withdrawn by the withdrawing of the catheter or sleeve.

As depicted in FIG. 28, which is a side view of a ninth example of a lead or catheter based platform on which an echo doppler sensor 15 may be supported to position the sensor 15 as discussed above with respect to FIG. 4, 8, 10, 12, 14 or 16, for example, the lead or catheter body 120 includes a pull wire 207 that causes the body 120 to deflect in the region of the body supporting the sensor 15. The pull wire 120 is fixed to the tubular body 120 at a fixation point 215 distal of the body deflection region 220. The pull wire is coupled to the tubular body 120 proximal of the deflection region 220 in such a manner so as to allow the pull wire to displace relative to the tubular body 120. Thus, pulling the wire 207 causes the portion of the body 120 immediately distal of the region 220 to move towards the portion of the body 120 immediately proximal of the region 220. As a result, the body 120 deflects into a bend in the region 220 and away from the wire 207, as indicated in FIG. 28.

As can be understood from FIG. 28, the bend region 220 of the body 120 ends up being configured to cause the deflected tubular body 120 to abut against the walls of the surrounding cardiac structure (e.g., SVC, right atrium, RVOT, CS, great cardiac vein, or etc.) with sufficient force to maintain the device 10 in position as indicated in FIG. 4, 8, 10, 12, 14 or 16. In other words, once the device 10 is delivered to the volume of a desired cardiac structure via a delivery tool, pulling the pull wire 207 causes the tubular body 120 to deflect into the configuration depicted in FIG. 28.

As illustrated in FIG. 28, in one embodiment, the echo Doppler sensor 15 is supported in or on the tubular body 120 at the midpoint or peak of the deflected portion 220 on the outside of the curve of the deflected portion 220. Thus, when the tubular body is implanted in the volume of the desired cardiac structure, the sensor 15 will be positioned in such an orientation to allow the sensor 15 to sense flow velocity across the a targeted flow region, such as, for example, the aortic root 65 (see FIG. 4), the mitral annulus 80 (see FIG. 8), the left anterior descending artery 90 (see FIG. 10), or the aorta 100 (see FIG. 12, 14 or 16).

In one embodiment, the device 10 of FIG. 28 is an implantable lead and, as a result, the sensor 15 may be permanently implanted at the desired monitoring location. Alternatively, the device 10 of FIG. 28 is a catheter or sheath and is used to temporarily locate the sensor 15 at the desired monitoring location. The catheter or sleeve is withdrawn from the monitoring location when the monitoring is completed, the sensor being withdrawn by the withdrawing of the catheter or sleeve.

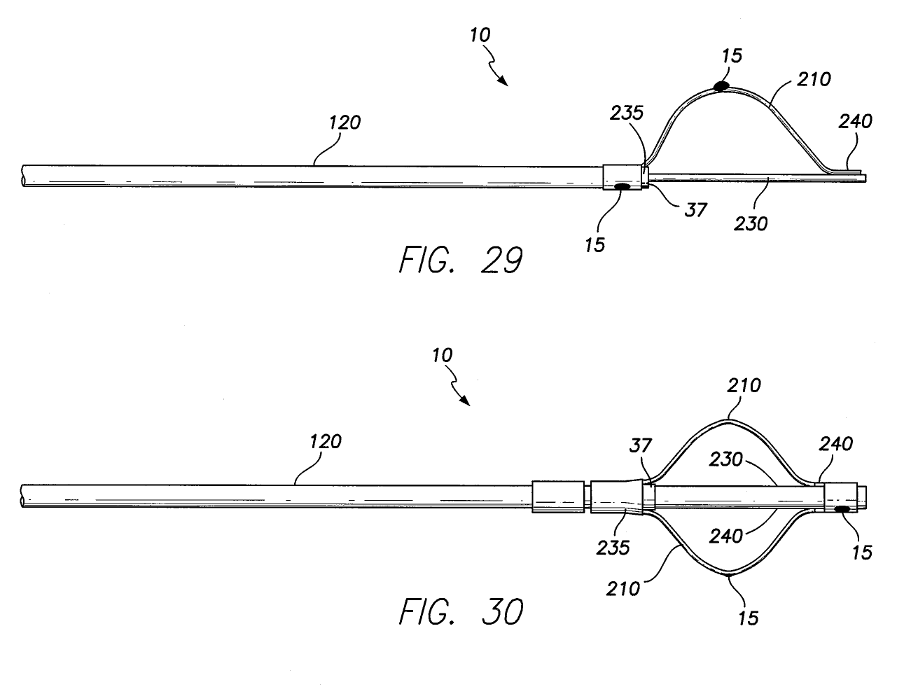

As shown in FIGS. 29 and 30, which are side views of two versions of a tenth example of a lead or catheter based platform on which an echo doppler sensor 15 may be supported to position the sensor 15 as discussed above with respect to FIG. 4, 8, 10, 12, 14 or 16, for example, the lead or catheter body 120 includes one or more deflection arms 210 and a pull member 230 that causes the arms 210 to deflect outwardly from the body 120. The pull member 230, which may be a pull wire or rod 230 (see FIG. 29) or an inner tubular body 230 (see FIG. 30), extends through the outer tubular body 120 and is displaceable distal-proximal within the outer tubular body 120. The proximal end 235 of each arm 210 is fixed to the tubular body 120 near the body distal end 37, and the distal end 240 of each arm 210 is fixed to the pull member 230 distal the outer tubular body distal end 37.

The pull member 230 is coupled within the outer tubular body 120 in such a manner so as to allow the pull member to displace proximal-distal relative to the tubular body 120. Thus, proximally pulling the member 230 causes the portion of the member 230 distal the body distal end 37 to move proximally towards the body distal end 37. As a result, the arms 210 deflect into bends and away from the member 230, as indicated in FIGS. 29 and 30.

As can be understood from FIGS. 29 and 30, the deflected arms 210 are configured to cause the deflected arms (or combination of deflected arm(s) and tubular body 120) to abut against the walls of the surrounding cardiac structure (e.g., SVC, right atrium, RVOT, CS, great cardiac vein, or etc.) with sufficient force to maintain the device 10 in position as indicated in FIG. 4, 8, 10, 12, 14 or 16. In other words, once the device 10 is delivered to the volume of a desired cardiac structure via a delivery tool, pulling the member 230 causes the arms 210 to deflect into the configurations depicted in FIGS. 29 and 30.

As illustrated in FIGS. 29 and 30, in one embodiment, the echo Doppler sensor 15 is supported in or on the tubular body 120 or at the midpoint or peak of a deflected arm 210 on the outside of the curve of the deflected arm 210. Thus, when the tubular body is implanted in the volume of the desired cardiac structure, the sensor 15 will be positioned in such an orientation to allow the sensor 15 to sense flow velocity across the a targeted flow region, such as, for example, the aortic root 65 (see FIG. 4), the mitral annulus 80 (see FIG. 8), the left anterior descending artery 90 (see FIG. 10), or the aorta 100 (see FIG. 12, 14 or 16).

In one embodiment, the device 10 of FIGS. 29 and 30 is an implantable lead and, as a result, the sensor 15 may be permanently implanted at the desired monitoring location. Alternatively, the device 10 of FIGS. 29 and 30 is a catheter or sheath and is used to temporarily locate the sensor 15 at the desired monitoring location. The catheter or sleeve is withdrawn from the monitoring location when the monitoring is completed, the sensor being withdrawn by the withdrawing of the catheter or sleeve.

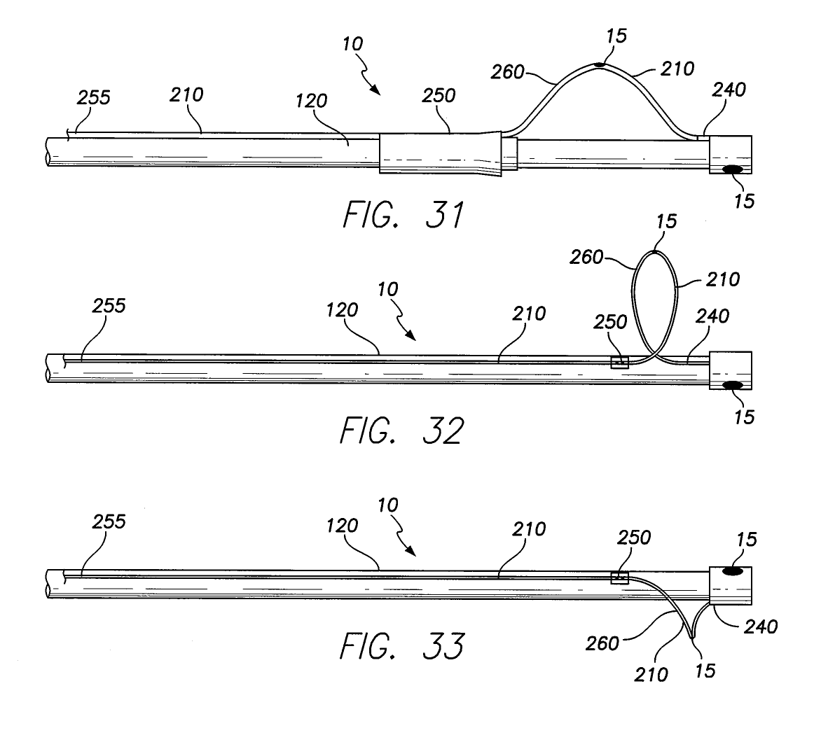

As shown in FIGS. 31, 32 and 33, which are side views of three versions of an eleventh example of a lead or catheter based platform on which an echo doppler sensor 15 may be supported to position the sensor 15 as discussed above with respect to FIG. 4, 8, 10, 12, 14 or 16, for example, the lead or catheter body 120 includes one or more deflection arms 210 that can be pushed distally relative to the body 120, causing the arms 210 to deflect outwardly from the body 120. The arms 210 may be formed of wire or another longitudinally extending member. The distal end 240 of each arm 210 is fixed to the tubular body 120 (e.g., near the body distal end 37). Proximally offset from the distal fixation point of each arm 210 is a retainer 250 that retains the arm 210 against the tubular body 120 at the location of the retainer, but allows the arm 210 to displace distally relative to the tubular body 120. Thus, distally pushing the proximal ends 255 of the arm(s) 210 causes the portion(s) 260 of the arm(s) 210 between the arm distal end(s) 240 and the retainer(s) 250 to deflect into bend(s) and away from the body 120, as indicated in FIGS. 31, 32 and 33.

As can be understood from FIGS. 31, 32 and 33, the deflected portions 260 of the arms 210 are configured to cause the deflected arms (or combination of deflected arm(s) and tubular body 120) to abut against the walls of the surrounding cardiac structure (e.g., SVC, right atrium, RVOT, CS, great cardiac vein, or etc.) with sufficient force to maintain the device 10 in position as indicated in FIG. 4, 8, 10, 12, 14 or 16. In other words, once the device 10 is delivered to the volume of a desired cardiac structure via a delivery tool, distally pushing the proximal arm ends 255 causes the arm portions 260 to deflect into the configurations depicted in FIGS. 31, 32 and 33.

As illustrated in FIGS. 31, 32 and 33, in one embodiment, the echo doppler sensor 15 is supported in or on the tubular body 120 or at the midpoint or peak of a deflected arm portion 260 on the outside of the curve of the deflected arm 210. Thus, when the tubular body is implanted in the volume of the desired cardiac structure, the sensor 15 will be positioned in such an orientation to allow the sensor 15 to sense flow velocity across the a targeted flow region, such as, for example, the aortic root 65 (see FIG. 4), the mitral annulus 80 (see FIG. 8), the left anterior descending artery 90 (see FIG. 10), or the aorta 100 (see FIG. 12, 14 or 16).

As shown in FIG. 31, the deflected arm 210 may assume a parabolic shape that generally fits into a single plane. As shown in FIG. 32, the deflected arm 210 may assume a loop. As shown in FIG. 33, the deflected arm 210 may assume a parabolic shape that does not fit in a single plane.

In one embodiment, the device 10 of FIGS. 31, 32 and 33 is an implantable lead and, as a result, the sensor 15 may be permanently implanted at the desired monitoring location. Alternatively, the device 10 of FIGS. 31, 32 and 33 is a catheter or sheath and is used to temporarily locate the sensor 15 at the desired monitoring location. The catheter or sleeve is withdrawn from the monitoring location when the monitoring is completed, the sensor being withdrawn by the withdrawing of the catheter or sleeve. The stent may also be of a material that is not self expanding but a material that can be permanently expanded using a balloon catheter or other tool.

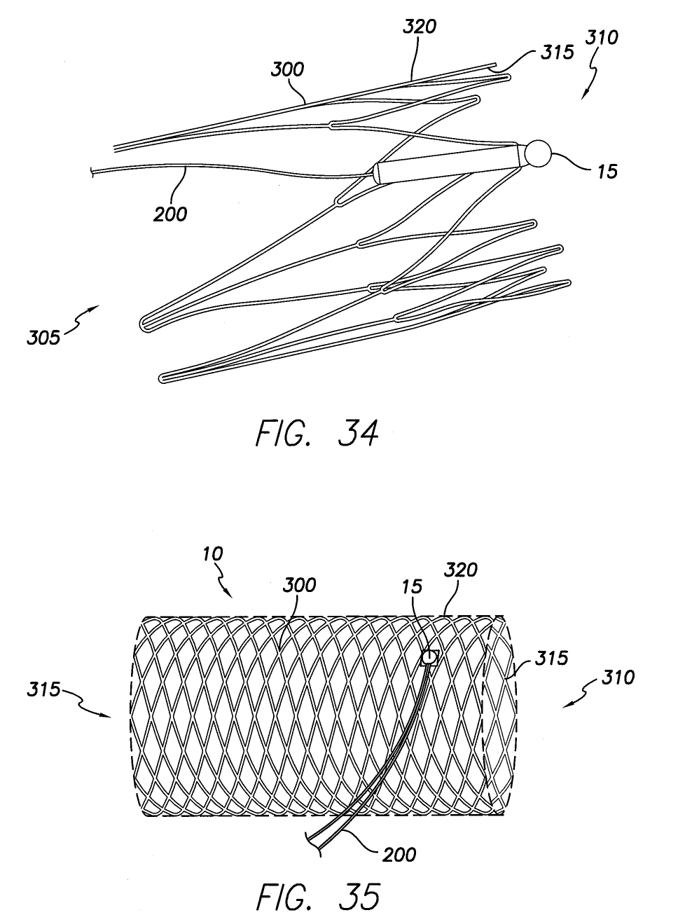

As shown in FIGS. 34 and 35, which are, respectively, side views of two versions of an example of a stent based platform on which an echo doppler sensor 15 may be supported to position the sensor 15 as discussed above with respect to FIG. 1, 4, 8, 10, 12, 14 or 16, for example, the implantable device 10 includes a self-expanding stent 300. In one version, as depicted in FIG. 34, the stent 300 is laser cut or otherwise formed from a tube, which may be formed of, for example, Nitinol or other materials having similar properties. In another version, as illustrated in FIG. 35, the stent 300 is a tubular braid or mesh, which may be formed of, for example, Nitinol or other materials having similar properties. In either case, the stents 300 of FIGS. 34 and 35 can be configured to be self-expanding such that the stent will bias into the expanded states depicted in FIGS. 34 and 35 once freed from the confines of a delivery tool such as, for example, a delivery catheter or sheath.

As indicated in FIGS. 34 and 35, the stents 300 include a proximal end 305, a distal end 310, an inner circumferential boundary 315, and an outer circumferential boundary 320, thereby having an overall cylindrical shape. The sensor 15 is mounted on a circumferential boundary 315, 320 of the stent and includes one or more electrical conductors 200 extending proximally from the sensor 15.

As can be understood from FIGS. 34 and 35, when the stent 300 is in an expanded state, the outer circumferential boundary 320 of the stent abuts against the walls of the surrounding cardiac structure (e.g., SVC, right atrium, RVOT, CS, great cardiac vein, subclavian vein, or etc.) with sufficient force to maintain the device 10 in position as indicated in FIG. 1, 4, 8, 10, 12, 14 or 16. In other words, once the device 10 is delivered to the volume of a desired cardiac structure via a delivery tool, releasing the stent from the confines of the delivery tool allows the stent to bias into its expanded state depicted in FIGS. 34 and 35 such that the outer circumferential boundary of the stent abuts against the walls of the surrounding cardiac structure to fix the stent in place within the cardiac structure.

As illustrated in FIGS. 34 and 35, in one embodiment, the echo Doppler sensor 15 is supported in or on the stent 300. Thus, when the stent is implanted in the volume of the desired cardiac structure, the sensor 15 will be positioned in such an orientation to allow the sensor 15 to sense flow velocity across the a targeted flow region, such as, for example, the subclavian artery (see FIG. 1), the aortic root 65 (see FIG. 4), the mitral annulus 80 (see FIG. 8), the left anterior descending artery 90 (see FIG. 10), or the aorta 100 (see FIG. 12, 14 or 16).

While most of FIGS. 19-35 depict a device 10 with a single sensor 15 mounted thereon, in many instances a single device 10, regardless of its configuration, will have multiple sensors 15 mounted thereon. For example, multiple sensors 15 may be used on the length and/or circumference of the device 10 whether the device 10 is a lead, lead-like device, catheter, sheath or stent of any of the embodiments discussed above with respect to FIGS. 19-35. The multiple sensors 15 can allow the flow monitoring system to which the sensors 15 are coupled (or a physician) to identify and select the individual sensor 15 of the multiple sensors 15 on the device 10 that gives the best signal without repositioning the device 10 in the vasculature or cardiac volume in which the device 10 is located. In other words, the multiple sensors 15 on a single device 10 allows for electronic repositioning of the sensing capability of the device 10.

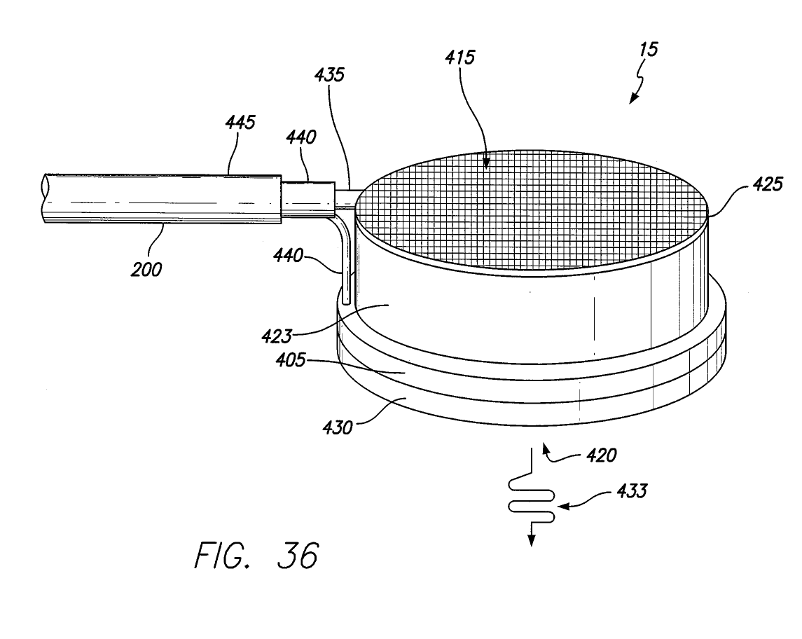

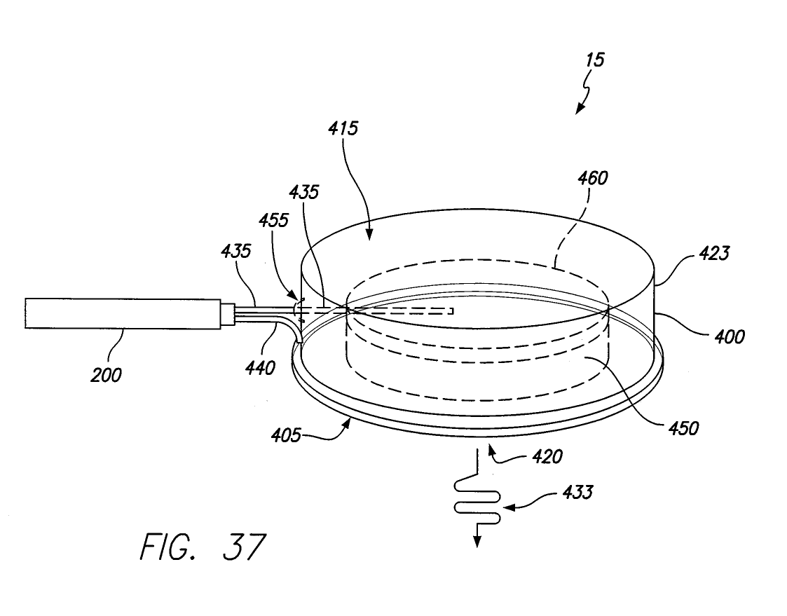

In one embodiment shown in FIGS. 36 and 37, the sensors 15 employed on the devices 10 employ a housing that is hermetically sealed that allows sensing but does not allow tissue or blood to contact the sensor. The material surrounding the sensor 15 is of appropriate acoustic impedance such that the signal can be eliminated from the non-sensing face and optimized for the sensing face. Optimization includes acoustic properties that allow for acoustic impedance transition from piezoelectric material to tissue and blood. This optimization allows for a high signal to noise ratio with a minimum amount of power consumption, thereby increasing battery life.

In some embodiments, the sensor and/or the sensing system to which the sensor is coupled employ an algorithm that allows the sensor and/or sensing system to monitor (power-up the sensor discretely) cardiac output one second every minute. Such an algorithm is useful, for example, in the case of monitoring congestive heart failure (“CHF”) progression and would considerably extend the life of the battery in a pacemaker or ICD to 25 years. The sensor could also be turned on during cardiac electrical anomalies such as a rapid heart rate. The blood flow monitoring system could then be used to distinguish ventricular tachycardia from ventricular fibrillation, reducing the number of inadvertent shocks and potentially increase the battery life of the pacemaker or ICD.

As discussed above, the system disclosed herein includes a sensor that can be implanted or placed in an arterial, venous or cardiac structure, the system being capable of measuring blood flow via the sensor in an adjacent arterial, venous or cardiac structure. The system includes a sensor, sensor housing, sensor fixation arrangement, and a signal generator/processor.

In one embodiment shown in FIGS. 36 and 37, the sensor is piezoelectric material (“PZT”) that is hermetically sealed in titanium and then coated with parylene. The sensor may be approximately 1 mm in diameter and have a thickness of approximately 0.1 mm. The sensor may have other configurations, such as, for example, a funnel or cone shape for use as part of an RV lead to measure flow through the tricuspid annulus. The funnel or cone shaped sensor would read flow in all directions around the lead, thus eliminating the need for directing the sensor beam.

In the case of a disk shaped sensor, the non-sensing face of the sensor is covered with a low acoustic impedance material such as, for example, expanded PTFE. The low acoustic material prevents signal from being transmitted from or entering into the non-sensing face of the disk.

The sensor is encased in titanium to eliminate the likelihood of the sensor coming in direct contact with the body. Piezoelectric materials may contain a small amount of lead. Therefore, direct contact with the body is not desirable.

The sensor has wires attached to each face allowing electrical current to activate the PZT creating an acoustic signal that is transmitted and received. The sensor emanates signal packets (bursts) at a known frequency. The signals emanating from the sensor are reflected off the moving blood and transmitted back to the sensor. The changes in frequency between the emitted and received signals can be analyzed to determine the velocity and direction of the blood flow. This phenomenon of frequency change as a result of motion is known as the Doppler Shift. By controlling the size of the sensor, matching the acoustic of the surrounding materials, input frequency and power to the sensor, the signal can be transmitted out to a desired distance. The sensor and system currently being tested have a sensing range of approximately 0 mm to approximately 10 mm.

As shown in FIG. 36, which is an isometric view of an example of a sensor 15 for use with any of the devices 10 discussed above with respect to FIGS. 19-35, the sensor 15 includes a housing 400 and an electrical conductor 200 extending therefrom. In one embodiment, the housing 400 is formed of titanium or another non-corrosive, biocompatible metal. The housing includes a flange 405 that facilitates welding of the housing to the device 10 and is formed of the same material as the rest of the housing (i.e., titanium). The housing is generally disk shaped and includes a non-sensing face 415, a sensing face 420 opposite the non-sensing face, and a circumferential side wall 423 separating the two faces 425, 420. The non-sensing face 415 includes a low acoustic impedance material 425 covering (i.e., generally coextensive with) the non-sensing face 415. The sensing face 420 includes parylene 430 or a similar material that covers (i.e., is generally coextensive with) the sensing face 420. The direction of acoustic transmission 433 extends from the sensing face 420.

A conductor 200 includes a signal portion 435 extending through the housing to be electrically coupled to the PZT housed in the housing 400 and a shield portion 440 electrically coupled to the housing 400 (e.g., at the flange 405). The conductor 200 may be in the form of a shielded coaxial cable wherein the signal portion 435 is the center conductor 435 of the coaxial cable and the shield portion 440 is the outer conductor 440 of the coaxial cable. The coaxial cable 200 includes an electrical insulation jacket 445.

As shown in FIG. 37, which is an isometric view of another example of a sensor 15 for use with any of the devices 10 discussed above with respect to FIGS. 19-35, the sensor 15 includes a housing 400 and an electrical conductor 200 extending therefrom. In one embodiment, the housing 400 is formed of titanium or another non-corrosive, biocompatible metal. The housing includes a flange 405 that facilitates welding of the housing to the device 10 and is formed of the same material as the rest of the housing (i.e., titanium). The housing is generally disk shaped and includes a non-sensing face 415, a sensing face 420 opposite the non-sensing face, and a circumferential side wall 423 separating the two faces 425, 420. The direction of acoustic transmission 433 extends from the sensing face 420.

A conductor 200 includes a signal portion 435 extending through the housing to be electrically coupled to the PZT 450 housed in the housing 400 and a shield portion 440 electrically coupled to the housing 400 (e.g., at the flange 405). A glass insulating feedthu 455 seals the penetration in the housing where the signal portion 435 extends through the housing. The conductor 200 may be in the form of a shielded coaxial cable wherein the signal portion 435 is the center conductor 435 of the coaxial cable and the shield portion 440 is the outer conductor 440 of the coaxial cable. The coaxial cable 200 includes an electrical insulation jacket 445.

A low acoustic impedance material 460 is enclosed by the housing 400 and covers (is generally coextensive with) the PZT 450. The entirety of the housing 400 is coated in parylene or a similar material. The signal portion 435 is coated in parylene or a similar material.

In one embodiment, the echo Doppler sensor 15 may have a housing configuration generally analogous to the housing configurations of the pressure sensors disclosed in U.S. Pat. Nos. 7,274,965, 7,340,288, 7,389,134, 7,448,999, and 7,450,999, wherein such pressure sensor housing configurations are adapted for use with an echo Doppler sensor as opposed to a pressure sensor. The disclosures of these patents are incorporated by reference into this Detailed Discussion in their entireties.

Although the present invention has been described with reference to preferred embodiments, persons skilled in the art will recognize that changes may be made in form and detail without departing from the spirit and scope of the invention.

What is claimed is:

1. A medical device comprising:
a tubular body including a distal end and a segment proximal to the distal end that is biased to assume a curved configuration; and
a blood flow velocity sensor supported on the segment,
wherein the tubular body is part of an implantable medical lead.

2. The device of claim 1, wherein the curved configuration includes at least two curved segments joined by a generally straight segment, the curved configuration generally existing in a single plane.

3. The device of claim 2, wherein the sensor is located on an outside of a curve of one of the at least two curved segments.

4. The device of claim 2, wherein the sensor is located on the generally straight segment.

5. The device of claim 2, wherein the sensor is located on a proximal side of the generally straight segment.

6. The device of claim 1, wherein the curved configuration is in the form of a helical coil.

7. The device of claim 6, wherein the sensor is located on an outside or inside of a curve of the helical coil.

8. A method of monitoring blood flow velocity, the method comprising:
providing a medical device comprising: a tubular body including a distal end and a segment proximal the distal end, the segment being capable of biasing into a curved configuration; and a blood flow velocity sensor supported on the segment, wherein the tubular body is part of an implantable medical lead, a catheter or sheath;
delivering the tubular body into a patient such that the sensor is located in a volume of a first venous, arterial or cardiac structure;
orienting the sensor to sense in the direction of a volume of a second venous, arterial or cardiac structure; and
allowing the segment to bias into the curved configuration to at least temporarily secure the sensor orientation.

9. The method of claim 8, wherein the first structure is a superior vena cava, right atrium, right atrial appendage, superior vena cava, or right ventricular outflow tract, and the second structure is a portion of the aorta.

10. The method of claim 8, wherein the first structure is a coronary sinus and the second structure a mitral annulus.

11. The method of claim 8, wherein the first structure is a great cardiac vein and the second structure a left descending artery.

12. A medical device comprising:
a tubular body including a distal end;
a fixation assembly near the distal end, the fixation assembly including a member deflectable away from the tubular body; and
a blood flow velocity sensor near the fixation assembly,
wherein the tubular body is part of an implantable medical lead, a catheter or sheath.

13. The device of claim 12, wherein the member is deflectable away from the tubular body via a self-biasing configuration of the member.

14. The device of claim 13, wherein the member includes a hinge like bend.

15. The device of claim 12, wherein the fixation assembly includes a proximally extending portion, which, when displaced longitudinally along the tubular body, causes the member to deflect away from the tubular body.

16. The device of claim 15, wherein the member includes a distal part fixed to the tubular body and a proximal part coupled to or extending into the proximally extending portion, distal displacement of the proximally extending portion causing the member to deflect away from the tubular body.

17. The device of claim 15, wherein the member includes a proximal part fixed to the tubular body near the distal end and a distal part coupled to or extending into the proximally extending portion, proximal displacement of the proximally extending portion causing the member to deflect away from the tubular body.

18. The device of claim 17, wherein the proximally extending portion extends longitudinally through the tubular body.

19. The device of claim 12, wherein the member includes a proximal part fixed to the tubular body and a distal part coupled to or extending into the proximally extending portion, proximal displacement of the proximally extending portion causing the member to deflect away from the tubular body.

20. A method of monitoring blood flow velocity, the method comprising:
providing a medical device comprising: a tubular body including a distal end; a fixation assembly near the distal end, the fixation assembly including a member deflectable away from the tubular body; and a blood flow velocity sensor near the fixation assembly, wherein the tubular body is part of an implantable medical lead, a catheter or sheath;
delivering the tubular body into a patient such that the sensor is located in a volume of a first venous, arterial or cardiac structure;
orienting the sensor to sense in the direction of a volume of a second venous, arterial or cardiac structure; and
causing the member to deflect away from the tubular body to at least temporarily secure the sensor orientation.

21. The method of claim 20, wherein the first structure is a superior vena cava, right atrium, right atrial appendage, superior vena cava, or right ventricular outflow tract, and the second structure is a portion of the aorta.

22. The method of claim 20, wherein the first structure is a coronary sinus and the second structure a mitral annulus.

23. The method of claim 20, wherein the first structure is a great cardiac vein and the second structure a left descending artery.

24. An implantable medical stent comprising:
an expandable body; and
a blood flow velocity sensor supported on the body.

25. The device of claim 24, wherein the body includes a mesh, braid or is formed via laser cutting from a cylinder.

26. A method of monitoring blood flow velocity, the method comprising:
providing an implantable medical stent comprising: an expandable body; and a blood flow velocity sensor supported on the body;
delivering the stent into a patient such that the sensor is located in a volume of a first venous, arterial or cardiac structure;
orienting the sensor to sense in the direction of a volume of a second venous, arterial or cardiac structure; and
causing the stent to expand to at least temporarily secure the sensor orientation.

27. The method of claim 26, wherein the first structure is a superior vena cava, right atrium, right atrial appendage, superior vena cava, or right ventricular outflow tract, and the second structure is a portion of the aorta.

28. The method of claim 26, wherein the first structure is a coronary sinus and the second structure a mitral annulus.

29. The method of claim 26, wherein the first structure is a great cardiac vein and the second structure a left descending artery.

30. A medical device comprising:
a tubular body including a distal end;
a deflection member extending longitudinally with the tubular body, wherein longitudinal displacement of the deflection member relative to the tubular body causes a segment of the tubular body to deflect into a curved configuration near the distal end; and
a blood flow velocity sensor supported on the segment,
wherein the tubular body is part of an implantable medical lead, a catheter or sheath.

31. The device of claim 30, wherein the deflection member includes: a distal part fixed to the tubular body near a distal region of the segment; and a proximal part displaceable relative to the tubular body, wherein proximal longitudinal displacement of the deflection member causes the segment of the tubular body to deflect into the curved configuration.

32. The device of claim 30, wherein the sensor is located on an outside of a curve of the segment in the curved configuration.

33. A method of monitoring blood flow velocity, the method comprising:
providing a medical device comprising: a tubular body including a distal end; a deflection member extending longitudinally with the tubular body, wherein longitudinal displacement of the deflection member relative to the tubular body causes a segment of the tubular body to deflect into a curved configuration near the distal end; and a blood flow velocity sensor supported on the segment, wherein the tubular body is part of an implantable medical lead, a catheter or sheath;
delivering the tubular body into a patient such that the sensor is located in a volume of a first venous, arterial or cardiac structure;
orienting the sensor to sense in the direction of a volume of a second venous, arterial or cardiac structure; and
causing the tubular body to deflect into the curved configuration to at least temporarily secure the sensor orientation.

34. The method of claim 33, wherein the first structure is a superior vena cava, right atrium, right atrial appendage, superior vena cava, or right ventricular outflow tract, and the second structure is a portion of the aorta.

35. The method of claim 33, wherein the first structure is a coronary sinus and the second structure a mitral annulus.

36. The method of claim 33, wherein the first structure is a great cardiac vein and the second structure a left descending artery.

37. An implantable medical lead comprising:
a tubular body including a distal end;
a suture sleeve on the tubular body; and
a blood flow velocity sensor near the suture sleeve.

38. The device of claim 37, wherein the sensor is supported on the sleeve.

39. The device of claim 37, wherein the sensor is supported on the tubular body.

40. A method of monitoring blood flow velocity, the method comprising:
providing a medical device comprising: a tubular body including a distal end; a suture sleeve on the tubular body; and a blood flow velocity sensor near the suture sleeve;
delivering the tubular body into a patient such that the sensor is located in a volume of a first venous or arterial structure;
orienting the sensor to sense in the direction of a volume of a second venous or arterial structure; and
securing the suture sleeve to the patient to secure the sensor orientation.

41. The method of claim 40, wherein the first structure is a subclavian vein, and the second structure is a subclavian artery.

42. The method of claim 40, wherein the first structure is a subclavian artery, and the second structure is a subclavian vein.

43. The method of claim 40, wherein the first structure is a vein, and the second structure is an adjacent artery.

44. The method of claim 40, wherein the first structure is an artery, and the second structure is an adjacent vein.

45. A method of monitoring blood flow velocity, the method comprising:
locating a blood flow velocity sensor near the ostium in the coronary sinus; and
sensing towards a portion of the aorta.

46. The method of claim 45, further comprising supporting the sensor on a lead, catheter, sheath or stent.

47. A method of monitoring blood flow velocity, the method comprising:
locating a blood flow velocity sensor in a vein; and
sensing towards an adjacent artery.

48. The method of claim 47, further comprising supporting the sensor on a lead, catheter, sheath or stent.

49. The method of claim 47, wherein the vein is a subclavian vein and the artery is a subclavian artery.

50. The method of claim 47, wherein the vein is a femoral vein and the artery is a femoral artery.

51. A method of monitoring blood flow velocity, the method comprising:
locating a blood flow velocity sensor near the tricuspid valve; and
sensing towards a tricuspid valve annulus.

52. The method of claim 51, further comprising supporting the sensor on a lead, catheter or sheath.

53. A method of monitoring blood flow velocity, the method comprising:
locating a blood flow velocity sensor right ventricular outflow tract; and
sensing towards a portion of the aorta.

54. The method of claim 53, further comprising supporting the sensor on a lead, catheter, sheath or stent.

55. A method of monitoring blood flow velocity, the method comprising:
locating a blood flow velocity sensor in the great cardiac vein; and
sensing towards a left anterior descending artery.

56. The method of claim 55, further comprising supporting the sensor on a lead, catheter, sheath or stent.

57. A method of monitoring blood flow velocity, the method comprising:
locating a blood flow velocity sensor in the right atrial appendage; and
sensing towards a portion of the aorta.

58. The method of claim 57, further comprising supporting the sensor on a lead, catheter, sheath or stent.

59. A medical device comprising:
a tubular body including a distal end; and
an echo doppler sensor supported on the tubular body and comprising: an acoustic transmission side; another side opposite the acoustic transmission side; a piezoelectric sensor; a titanium housing forming a side wall about the piezoelectric sensor and located between the acoustic transmission side and the another side; and a low acoustic impedance material on an opposite side of the piezoelectric sensor from the acoustic transmission side;
wherein the tubular body is part of an implantable medical lead, a catheter or sheath.

60. The device of claim 59, wherein the low acoustic impedance material is coextensive with a surface of the piezoelectric sensor adjacent the another side.

61. The device of claim 59, wherein the low acoustic impedance material is coextensive with the another side.

62. The device of claim 59, further comprising a parylene coating on the acoustic transmission side.

63. The device of claim 59, further comprising a parylene coating on the housing.

64. The device of claim 59, further comprising a coaxial cable, a shield layer of the cable being electrically coupled to the housing, and a center conductor of the cable being electrically coupled to the piezoelectric sensor.
